(19) United States
(12) Patent Application Publication
(10) Pub. No.: US 2011/0230407 A1
(43) Pub. Date: Sep. 22, 2011

(54) HEPATOCYTE GROWTH FACTOR PATHWAY ACTIVATORS IN DEMYELINATING DISEASES AND CENTRAL NERVOUS SYSTEM TRAUMA

(76) Inventor: Alexander Yuzhakov, Bronx, NY (US)

(21) Appl. No.: 13/118,317

(22) Filed: May 27, 2011

Related U.S. Application Data
(63) Continuation of application No. 11/374,552, filed on Mar. 13, 2006, now abandoned.
(60) Provisional application No. 60/661,637, filed on Mar. 14, 2005.

(51) Int. Cl. A61K 38/22; A61K 31/415; A61K 48/00; A61K 31/4155; A61K 31/4439; A61K 31/454; A61K 31/5377; A61K 31/42; A61K 31/4704; A61P 25/00

(52) U.S. Cl. 514/9.5; 514/406; 514/44 R; 514/341; 514/326; 514/236.5; 514/378; 514/312

(57) ABSTRACT

Methods are provided for treating and preventing demyelinating diseases including multiple sclerosis, and traumatic injury to the central nervous system including brain trauma and spinal cord injury, by administering a compound or pharmaceutical composition of the invention. Useful compounds include hepatocyte growth factor/scatter factor protein, fragments, fusion polypeptides and muteins thereof, and nucleic acid and expression vectors encoding such proteins. Other useful compounds include small molecule HGF/SF agonists and mimetics.

### PRIORITY

This application is a continuation of U.S. patent application Ser. No. 11/374,552, filed Mar. 13, 2006, which claims priority to provisional application Ser. No. 60/661,637, filed Mar. 14, 2005, both of which are incorporated herein by reference in their entireties.

### GOVERNMENT SUPPORT

This work was supported in part by the U.S. Government, grant 2R44DK062592 from the Public Health Service, National Institutes of Health. The U.S. Government may have certain rights to this invention.

### FIELD OF THE INVENTION

This invention relates generally to methods for treating and preventing diseases of the central nervous system, for example demyelinating diseases and central nervous system trauma, by administration of proteins, nucleic acids, or small molecules which activate signaling pathways that occur as a consequence of the binding of hepatocyte growth factor (scatter factor) to its cellular receptor, Met.

### BACKGROUND OF THE INVENTION

Diseases of the central nervous system such as demyelinating diseases, including multiple sclerosis, and trauma to the central nervous system, for example brain trauma and spinal cord injury, are devastating conditions for which effective treatments are limited or nonexistent. While the patient populations are not dramatic, the impact of these diseases and injuries on the patients' and their families' lives certainly are.

Demyelinating diseases are those in which myelin is the primary target. They fall into two main groups: acquired diseases (i.e., multiple sclerosis) and hereditary neurodegenerative disorders (i.e., the leukodystrophies). Although their causes and etiologies are different, they have the same outcome: central nervous system (CNS) demyelination. Without myelin, nerve impulses are slowed or stopped, leading to a constellation of neurological symptoms. Multiple sclerosis (MS) is the most common demyelinating disease, which usually manifests itself between the 20th and 50th years of life. Current estimates are that approximately 2.5 million people worldwide have MS, with between 250,000 and 350,000 cases in the United States, 50,000 cases in Canada, 130,000 cases in Germany, 85,000 cases in the United Kingdom, 75,000 cases in France, 50,000 cases in Italy, and 11,000 cases in Switzerland.

MS attacks the white matter of the CNS. In its classic manifestation (90% of all cases), it is characterized by alternating relapsing/remitting phases with the periods of remission growing shorter over time. Its symptoms include any combination of spastic paraparesis, unsteady gait, diplopia, and incontinence.

Other demyelinating diseases include leukodystrophies: metachromatic leukodystrophy, Refsum's disease, adrenoleukodystrophy, Krabbe's disease, phenylketonuria, Canavan disease, Pelizaeus-Merzbacher disease and Alexander's disease. The first six are storage disorders. The lack or the malfunctioning of an enzyme causes a toxic buildup of chemical substances. In Pelizaeus-Merzbacher disease myelin is never formed (dysmyelination) because of a mutation in the gene that produces a basic protein of CNS myelin. The etiology of Alexander's disease remains largely unknown.

Among traumatic injury to the central nervous system, spinal cord injury (SCI) occurs in about 11,000 individuals per year in the US. Patients with SCI, usually have permanent and often devastating neurologic deficits. The types of disability associated with SCI vary greatly depending on the severity of the injury, the segment of the spinal cord at which the injury occurs, and the precise nerve fibers damaged. Destruction of nerve fibers carrying motor signals from the brain to the torso and limbs leads to muscle paralysis. Destruction of sensory nerve fibers can lead to loss of sensations such as touch, pressure, and temperature. Other serious consequences can include exaggerated reflexes; loss of bladder and bowel control; sexual dysfunction; lost or decreased breathing capacity; impaired cough reflexes; and spasticity. Secondary damage to the spinal cord, which continues for some hours after initial SCI can cause loss of myelin, neuronal death and axonal degeneration. Currently, methods for reducing the extent of SCI and for restoring function are severely limited.

Brain injury including brain shear can occur in many ways. Traumatic brain injuries, the most common, typically result from accidents in which the head strikes an object. However, other brain injuries, such as those caused by insufficient oxygen, poisoning, or infection, can cause similar deficits. Traumatic brain injury (TBI) can significantly affect many cognitive, physical, and psychological skills. Physical deficit can include ambulation, balance, coordination, fine motor skills, strength, and endurance. Cognitive deficits of language and communication, information processing, memory, and perceptual skills are common. Psychological status is also often altered. Adjustment to disability issues are frequently encountered by people with TBI.

Mild Traumatic Brain Injury (MTBI) is characterized by one or more of the following symptoms: a brief loss of consciousness, loss of memory immediately before or after the injury, any alteration in mental state at the time of the accident, or focal neurological deficits. In many MTBI cases, the person seems fine on the surface, yet continues to endure chronic functional problems. Some people suffer long-term effects of MTBI, known as postconcussion syndrome (PCS). Persons suffering from PCS can experience significant changes in cognition and personality.

Scatter factor (SF; also known as hepatocyte growth factor [HGF], and hereinafter referred to and abbreviated as HGF, SF, or HGF/SF) is a pleiotropic growth factor that stimulates cell growth, cell motility, morphogenesis and angiogenesis. HGF/SF is produced as an inactive monomer (˜100 kDa) which is proteolytically converted to its active form. Active HGF/SF is a heparin binding heterodimeric protein composed of a 62 kDa α chain and a 34 kDa 0 chain. HGF/SF signals through the HGF/SF Receptor, Met. In vitro and in vivo studies have indicated that the multiple biological effects of HGF/SF are mediated by a signal cascade initiated by HGF/SF binding to its tyrosine kinase receptor, Met. Met is a heterodimeric receptor tyrosine kinase composed of a 45 kDa extracellular subunit and a transmembrane 145 kDa kinase catalytic domain, which are linked by disulfide bridges. Interaction of Met with HGF/SF leads to autophosphorylation of several tyrosine residues, which regulate the kinase activity of the receptor and serve as binding sites for downstream adaptor molecules.

As treatment modalities for the aforementioned conditions and diseases are quite limited, need exists for identifying new approaches to address them. The present invention is directed to methods of using compounds that activate hepatocyte growth factor/scatter factor (HGF/SF)-Met pathways and activities for the treatment, prevention or prophylaxis of diseases and conditions related to demyelinization or trauma to the central nervous system.

All citations in the present application are incorporated herein by reference in their entireties. The citation of any reference herein should not be construed as an admission that such reference is available as “Prior Art” to the instant application.

### SUMMARY OF THE INVENTION

It has been discovered that various agents and compounds that activate HGF/SF pathways are useful in the treatment and prophylaxis of various demyelinating diseases and traumatic diseases of the central nervous system. The present invention is directed generally to the treatment and prevention of various demyelinating diseases and conditions related thereto, such as but not limited to multiple sclerosis and various hereditary neurodegenerative diseases, and to sequelae occurring after traumatic injury to the central nervous system, such as spinal cord injury (SCI) and traumatic brain injury, using agents that activate HGF/SF signaling pathways. Agents that activate signaling pathways of hepatocyte growth factor (HGF, also known as scatter factor (SF), and abbreviated HGF, SF or HGF/SF) include HGF protein, active fragments thereof, HGF/SF muteins and active fragments thereof, HGF/SF fusion polypeptides; nucleic acids and expression vectors encoding HGF/SF, fragments thereof, HGF/SF muteins and fragments thereof, fusion polypeptides thereof, as well as small molecule HGF/SF agonists and mimetics. The invention embraces all such HGF/SF signaling pathway activators for the purposes described herein.

Among the small molecule activators described above for the uses herein, in one embodiment, compounds are substituted pyrazoles having the structure:

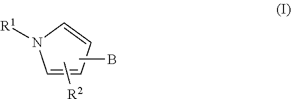

wherein R¹, R² and B are as described generally and in classes and subclasses herein.

In certain embodiments, the present invention provides novel compounds of general formula (II^{A1}) and (III^{D1}),

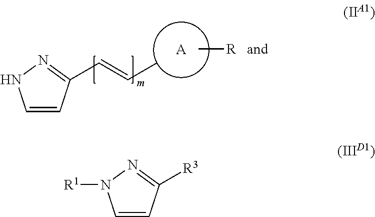

tautomers thereof, C(5)-positional isomers thereof; and pharmaceutical compositions thereof, as described generally and in subclasses herein, which compounds are useful as modulators of HGF/SF activity.

In other embodiments, compounds useful in the practice of the invention include those with the general formulae:

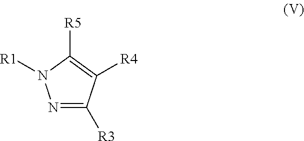

wherein R3 and R5 are independently or together a straight-chain or branched C₁₋₆ alkyl optionally substituted with a cyano or halogen, halogen, trifluoromethyl or difluoromethyl groups; R1 is hydrogen, methyl, CO-Aryl, SO₂-Aryl, CO-heteroaryl, or CO-alkyl; and R4 is CH₂-Aryl, halogen, arylcarbonylvinyl or S-heteroaryl; and

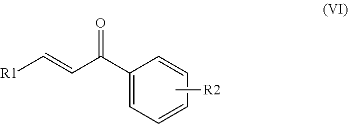

wherein R1 is Aryl or Heteroaryl; and R2 is one or more halogen, nitro, C₁₋₄ straight-chained alkyl, branched alkyl, or cycloalkyl, or C₁₋₄ alkyloxy groups.

### BRIEF DESCRIPTIONS OF THE FIGURES

FIG. 1 shows the effect of HGF/SF and a HGF/SF mimetic on hydrogen peroxide-induced apoptosis of Schwann cells in vitro.

FIGS. 2 A-B show the effect of HGF/SF and a mimetic on myelin production in Schwann cells and oligodendrocytes.

FIGS. 3 A-D show the effect of HGF/SF and a mimetic on Schwann cell and oligodendrocyte proliferation.

FIGS. 4 A-B show the effect of HGF/SF and a mimetic on phosphorylation of Met and Erk.

FIG. 5 A-D show the effect of HGF/SF and a mimetic on neuronal cell proliferation.

FIG. 6 shows the effect of HGF/SF and a mimetic on expression of neurotropic factors by Schwann cells.

FIG. 7 shows the HGF/SF and a mimetic on activating Schwann cell migration.

FIG. 8 shows the effect of a HGF/SF mimetic on a multiple sclerosis model.

FIGS. 9 A-B show the effect of a HGF/SF mimetic on spinal cord injury.

### DEFINITIONS

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, or alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having 1-6 carbon atoms. “Lower alkenyl” and “lower alkynyl” respectively include corresponding 1-6 carbon moieties.

In certain embodiments, the alkyl, alkenyl and alkynyl groups employed in the invention contain 1-20; 2-20; 3-20; 4-20; 5-20; 6-20; 7-20 or 8-20 aliphatic carbon atoms. In certain other embodiments, the alkyl, alkenyl, and alkynyl groups employed in the invention contain 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl, alkenyl, and alkynyl groups employed in the invention contain 1-6; 2-6; 3-6; 4-6 or 5-6 aliphatic carbon atoms. In yet other embodiments, the alkyl, alkenyl, and alkynyl groups employed in the invention contain 1-4; 2-4 or 3-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl(propargyl), 1-propynyl and the like.

The term “alicyclic”, as used herein, refers to compounds which combine the properties of aliphatic and cyclic compounds and include but are not limited to monocyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl, cyclohexyl, —CH₂-cyclohexyl, cyclohexenylethyl, cyclohexanylethyl, norborbyl moieties and the like, which again, may bear one or more substituents.

The term “alkoxy” or “alkyloxy”, as used herein refers to a saturated (i.e., O-alkyl) or unsaturated (i.e., O-alkenyl and O-alkynyl) group attached to the parent molecular moiety through an oxygen atom. In certain embodiments, the alkyl group contains 1-20; 2-20; 3-20; 4-20; 5-20; 6-20; 7-20 or 8-20 aliphatic carbon atoms. In certain other embodiments, the alkyl group contains 1-10; 2-10; 3-10; 4-10; 5-10; 6-10; 7-10 or 8-10 aliphatic carbon atoms. In yet other embodiments, the alkyl, alkenyl, and alkynyl groups employed in the invention contain 1-8; 2-8; 3-8; 4-8; 5-8; 6-20 or 7-8 aliphatic carbon atoms. In still other embodiments, the alkyl group contains 1-6; 2-6; 3-6; 4-6 or 5-6 aliphatic carbon atoms. In yet other embodiments, the alkyl group contains 1-4; 2-4 or 3-4 aliphatic carbon atoms. Examples of alkoxy, include but are not limited to, methoxy, ethoxy, propoxy, isopropoxy, n-butoxy, i-butoxy, sec-butoxy, tert-butoxy, neopentoxy, n-hexoxy and the like.

The term “thioalkyl” as used herein refers to a saturated (i.e., S-alkyl) or unsaturated (i.e., S-alkenyl and S-alkynyl) group attached to the parent molecular moiety through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of thioalkyl include, but are not limited to, methylthio, ethylthio, propylthio, isopropylthio, n-butylthio, and the like.

The term “alkylamino” refers to a group having the structure —NHR′ wherein R′ is aliphatic or alicyclic, as defined herein. The term “aminoalkyl” refers to a group having the structure NH₂R′-, wherein R′ is aliphatic or alicyclic, as defined herein. In certain embodiments, the aliphatic or alicyclic group contains 1-20 aliphatic carbon atoms. In certain other embodiments, the aliphatic or alicyclic group contains 1-10 aliphatic carbon atoms. In still other embodiments, the aliphatic or alicyclic group contains 1-6 aliphatic carbon atoms. In yet other embodiments, the aliphatic or alicyclic group contains 1-4 aliphatic carbon atoms. In yet other embodiments, R′ is an alkyl, alkenyl, or alkynyl group containing 1-8 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SR_{X}; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aryl or heteroaryl substituents described above and herein may be substituted or unsubstituted. Additional examples of generally applicable substituents are illustrated by the specific embodiments shown in the Examples that are described herein.

In general, the term “aromatic moiety”, as used herein, refers to a stable mono- or polycyclic, unsaturated moiety having preferably 3-14 carbon atoms, each of which may be substituted or unsubstituted. In certain embodiments, the term “aromatic moiety” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”.

In general, the term “heteroaromatic moiety”, as used herein, refers to a stable mono- or polycyclic, unsaturated moiety having preferably 3-14 carbon atoms, each of which may be substituted or unsubstituted; and comprising at least one heteroatom selected from O, S and N within the ring (i.e., in place of a ring carbon atom). In certain embodiments, the term “heteroaromatic moiety” refers to a planar ring comprising at least one heteroatom, having p-orbitals perpendicular to the plane of the ring at each ring atom, and satisfying the Huckel rule where the number of pi electrons in the ring is (4n+2) wherein n is an integer.

It will also be appreciated that aromatic and heteroaromatic moieties, as defined herein may be attached via an alkyl or heteroalkyl moiety and thus also include -(alkyl)aromatic, -(heteroalkyl)aromatic, -(heteroalkyl)heteroaromatic, and -(heteroalkyl)heteroaromatic moieties. Thus, as used herein, the phrases “aromatic or heteroaromatic moieties” and “aromatic, heteroaromatic, -(alkyl)aromatic, -(heteroalkyl)aromatic, -(heteroalkyl)heteroaromatic, and -(heteroalkyl)heteroaromatic” are interchangeable. Substituents include, but are not limited to, any of the previously mentioned substituents, i.e., the substituents recited for aliphatic moieties, or for other moieties as disclosed herein, resulting in the formation of a stable compound.

The term “aryl”, as used herein, does not differ significantly from the common meaning of the term in the art, and refers to an unsaturated cyclic moiety comprising at least one aromatic ring. In certain embodiments, “aryl” refers to a mono- or bicyclic carbocyclic ring system having one or two aromatic rings including, but not limited to, phenyl, naphthyl, tetrahydronaphthyl, indanyl, indenyl and the like.

The term “heteroaryl”, as used herein, does not differ significantly from the common meaning of the term in the art, and refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SR_{X}; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SR_{X}; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be linear or branched, and saturated or unsaturated. In certain embodiments, heteroaliphatic moieties are substituted by independent replacement of one or more of the hydrogen atoms thereon with one or more moieties including, but not limited to aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SR_{X}; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heterocycloalkyl”, “heterocycle” or “heterocyclic”, as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from O, S and N (wherein the nitrogen and sulfur heteroatoms may be optionally be oxidized), wherein the ring systems are optionally substituted with one or more functional groups, as defined herein. In certain embodiments, the term “heterocycloalkyl”, “heterocycle” or “heterocyclic” refers to a non-aromatic 5-, 6- or 7-membered ring or a polycyclic group wherein at least one ring atom is a heteroatom selected from O, S and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, heterocycles such as furanyl, thiofuranyl, pyranyl, pyrrolyl, pyrazolyl, imidazolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(═O)Rₓ; —CO₂(Rₓ); —C(═O)N(Rₓ)₂; —OC(═O)Rₓ; —OCO₂Rₓ; —OC(═O)N(Rₓ)₂; —N(Rₓ)₂; —ORₓ; —SR_{X}; —S(O)Rₓ; —S(O)₂Rₓ; —NRₓ(CO)Rₓ; —N(Rₓ)CO₂Rₓ; —N(Rₓ)S(O)₂Rₓ; —N(Rₓ)C(═O)N(Rₓ)₂; —S(O)₂N(Rₓ)₂; wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples or generally applicable substituents are illustrated by the specific embodiments shown in the Examples, which are described herein.

Additionally, it will be appreciated that any of the alicyclic or heterocyclic moieties described above and herein may comprise an aryl or heteroaryl moiety fused thereto. Additional examples of generally applicable substituents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “amino”, as used herein, refers to a primary (—NH₂), secondary (—NHRₓ), tertiary (—NRₓR_{y}) or quaternary (—N⁺RₓR_{y}R_{z}) amine, where Rₓ, R_{y} and R^{z}, are independently an aliphatic, alicyclic, heteroaliphatic, heterocyclic, aromatic or heteroaromatic moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

The term “acyl”, as used herein, refers to a group having the general formula —C(═O)R, where R is an aliphatic, alicyclic, heteroaliphatic, heterocyclic, aromatic or heteroaromatic moiety, as defined herein.

The term “C₂₋₆alkenylidene”, as used herein, refers to a substituted or unsubstituted, linear or branched unsaturated divalent radical consisting solely of carbon and hydrogen atoms, having from two to six carbon atoms, having a free valence “-” at both ends of the radical, and wherein the unsaturation is present only as double bonds and wherein a double bond can exist between the first carbon of the chain and the rest of the molecule.

As used herein, the terms “aliphatic”, “heteroaliphatic”, “alkyl”, “alkenyl”, “alkynyl”, “heteroalkyl”, “heteroalkenyl”, “heteroalkynyl”, and the like encompass substituted and unsubstituted, saturated and unsaturated, and linear and branched groups. Similarly, the terms “alicyclic”, “heterocyclic”, “heterocycloalkyl”, “heterocycle” and the like encompass substituted and unsubstituted, and saturated and unsaturated groups. Additionally, the terms “cycloalkyl”, “cycloalkenyl”, “cycloalkynyl”, “heterocycloalkyl”, “heterocyclealkenyl”, “heterocycloalkynyl”, “aromatic”, “heteroaromatic”, “aryl”, “heteroaryl” and the like encompass both substituted and unsubstituted groups.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Another example is an N-methyl derivative of a compound, which is susceptible to oxidative metabolism resulting in N-demethylation, particularly on the 1 position of the 3(5)-monosubstituted pyrazole derivatives of the invention. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

The term “tautomerization” refers to the phenomenon wherein a proton of one atom of a molecule shifts to another atom. See, Jerry March, Advanced Organic Chemistry: Reactions, Mechanisms and Structures, Fourth Edition, John Wiley & Sons, pages 69-74 (1992). The term “tautomer” as used herein, refers to the compounds produced by the proton shift. For example, compounds of formula II (and more generally, compounds of formula I where R¹ is hydrogen), can exist as a tautomer as shown below:

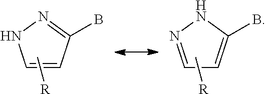

Thus, the present invention encompasses the 3-monosubstituted pyrazole compounds described herein, as well as their tautomeric 5-monosubstituted pyrazole counterparts. Likewise, any compound shown as 5-monosubstituted pyrazole embraces its corresponding 3-monosubstituted tautomer. The term “C(5)-positional isomer” as used herein refers to 1,5-disubstituted counterparts of the 1,3-disubstituted pyrazole compounds described herein. For example, the invention encompasses compounds of the formula (III^{B}) and its C(5)-positional isomer (III^{B}):

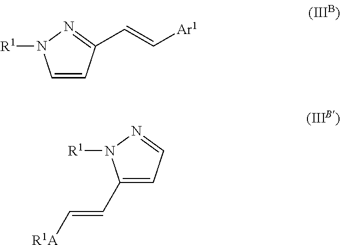

Thus, whether or not explicitly specified, the present invention encompasses the 1,3-disubstituted pyrazole compounds described herein (e.g., compounds of formula I, III, and related formulae III^{A}, III^{B}, III^{C}, III^{D}, etc. . . . ), as well as their C(5)-positional pyrazole counterparts. Likewise, any compound shown as 1,5-disubstituted pyrazole embraces its corresponding 1,3-disubstituted positional isomer.

By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

The term “hepatocyte growth factor/scatter factor pathway activator” refers to a compound or agent that induces or initiates the signaling cascade from Met, the HGF/SF receptor. Such compounds other than HGF/SF are also referred to as HGF/SF mimetics or agonists. Examples include small molecule HGF/SF agonists or mimetics, or HGF/SF protein or active fragments, fusion polypeptides, or muteins thereof. The term also refers to a compound or agent that induces the expression of a molecule that induces or initiates the signaling cascade from Met, the HGF/SF receptor, such as DNA or an expression vector that upon administration and incorporation of the DNA or vector with cells, induces the expression of HGF/SF or an active fragment, fusion polypeptide or mutein thereof.

### DETAILED DESCRIPTION OF CERTAIN PREFERRED EMBODIMENTS OF THE INVENTION

The present invention is directed generally to the treatment and prevention of various demyelinating diseases and conditions related thereto, such as but not limited to multiple sclerosis and various neurodegenerative diseases, and to sequelae occurring after traumatic injury to the central nervous system, such as spinal cord injury and traumatic brain injury. Agents that activate signaling pathways of hepatocyte growth factor (also known as scatter factor; abbreviated HGF or HGF/SF) such as HGF protein, fragments, fusion polypeptides and muteins or other active variants thereof, and nucleic acids and expression vectors encoding HGF, fragments, fusion polypeptides and muteins thereof, as well as small molecule HGF agonists and mimetics, have been found to be useful in various therapeutic modalities including prophylaxis and treatment of the aforementioned pathologies. Such agents and compounds are described in the following documents, all of which are incorporated herein in their entireties: 1) hepatocyte growth factor/scatter factor protein, such as is described in U.S. Pat. Nos. 5,837,676; 5,919,759; 5,965,523; 6,011,009; 6,013,624; 6,498,144; 6,699,837; and 6,303,126; 2) hepatocyte growth factor/scatter factor protein fragments, fusion proteins or polypeptides, muteins, truncates, and other modified forms of HGF, such as by way of non-limiting example are described in U.S. Pat. No. 6,566,098, that have the ability to activate HGF pathways; 3) nucleic acid encoding HGF/SF or HGF/SF muteins or those agents and compounds described in (2) above, such as described in U.S. Pat. Nos. 6,248,722, 6,258,787 and 6,566,098; and 4) small molecule HGF/SF pathway activators, such as but not limited to: a) compounds described in published U.S. patent applications US20040180882, US20050113369, US20050192331 and in PCT/US03/40917 published as WO2004/058721; and b) compounds described in U.S. Pat. Nos. 6,589,997, 6,610,726, 6,855,728, and PCT/US01/20849, published as WO02/002593. The foregoing are merely illustrative of HGF/SF pathway activators useful in the practice of the present invention, yet they are not limiting.

Demyelinating diseases are those in which myelin is the primary target. They fall into two main groups: acquired diseases (i.e., multiple sclerosis) and hereditary neurodegenerative disorders (i.e., the leukodystrophies). Although their causes and etiologies are different, they have the same outcome: CNS demyelination. Without myelin, nerve impulses are slowed or stopped, leading to a constellation of neurological symptoms.

Multiple Sclerosis. The most common of these is multiple sclerosis (MS), which usually manifests itself between the 20th and 50th years of life. Current estimates are that approximately 2.5 million people worldwide have MS, with between 250,000 and 350,000 cases in the United States, 50,000 cases in Canada, 130,000 cases in Germany, 85,000 cases in the United Kingdom, 75,000 cases in France, 50,000 cases in Italy, and 11,000 cases in Switzerland.

MS attacks the white matter of the central nervous system (CNS). In its classic manifestation (90% of all cases), it is characterized by alternating relapsing/remitting phases with periods of remission growing shorter over time. Its symptoms include any combination of spastic paraparesis, unsteady gait, diplopia, and incontinence.

Hereditary Neurodegenerative Disorders. This category includes the eight identified leukodystrophies: metachromatic leukodystrophy, Refsum's disease, adrenoleukodystrophy, Krabbe's disease, phenylketonuria, Canavan disease, Pelizaeus-Merzbacher disease and Alexander's disease. The first six are storage disorders. The lack or the malfunctioning of an enzyme causes a toxic buildup of chemical substances. In Pelizaeus-Merzbacher disease myelin is never formed (dysmyelination) because of a mutation in the gene that produces a basic protein of CNS myelin. The etiology of Alexander's disease remains largely unknown.

The clinical course of hereditary demyelinating disorders, which usually tend to manifest themselves in infancy or early childhood, is tragic. Previously normal children are deprived, in rapid progression, of sight, hearing, speech, and ambulation. Equally tragic is their prognosis: death within a few years.

Spinal cord injury (SCI). The number of people in the United States currently living with SCI is estimated to be between 721 and 906 per million population. This corresponds to between 183,000 and 230,000 persons. Patients with SCI, usually have permanent and often devastating neurologic deficits. The types of disability associated with SCI vary greatly depending on the severity of the injury, the segment of the spinal cord at which the injury occurs, and the precise nerve fibers damaged. Destruction of nerve fibers carrying motor signals from the brain to the torso and limbs leads to muscle paralysis. Destruction of sensory nerve fibers can lead to loss of sensations such as touch, pressure, and temperature. Other serious consequences can include exaggerated reflexes; loss of bladder and bowel control; sexual dysfunction; lost or decreased breathing capacity; impaired cough reflexes; and spasticity. Many patients with SCI regain some functions between one week and six months after injury, but the likelihood of spontaneous recovery diminishes after six months. In addition, SCI may result in secondary complications, including pressure sores, increased susceptibility to respiratory diseases, and autonomic dysreflexia (a potentially life-threatening increase in blood pressure, sweating, and other autonomic reflexes in reaction to bowel impaction or some other stimulus). The most common types of spinal cord injuries are contusions (bruising of the spinal cord) and compression injuries (caused by pressure on the spinal cord) resulting in neuronal de-myelination. Other types of injury included lacerations, caused by a bullet or other object, and central cord syndrome which affects the cervical region of the cord and results from focused damage to the corticospinal tract. Complete severing of the spinal cord is rare in humans. Secondary damage to the spinal cord, which continues for some hours after initial SCI can cause loss of myelin, neuronal death and axonal degeneration. Currently, methods for reducing the extent of SCI and for restoring function are severely limited.

Current therapy for recovery from SCI. Presently, treatment of acute SCI involves: diagnosing and relieving gross misalignments and other structural problems of the spine, minimizing cellular-level damage (methylprednisolone), and stabilizing the vertebrae to prevent further injury. Once a patient is stabilized, supportive care and rehabilitation strategies promote long-term recovery. Neural prostheses present an important approach for improving the quality of life after trauma. Recent advances in the clinical management of SCI include improved imaging of damage to the spinal cord and vertebrae as well as development of methylprednisolone as the first effective drug therapy for use in the hours just after injury, when secondary damage can occur.

Trauma to the Brain. Traumatic brain injury (TBI) can significantly affect many cognitive, physical, and psychological skills. Physical deficit can include ambulation, balance, coordination, fine motor skills, strength, and endurance. Cognitive deficits of language and communication, information processing, memory, and perceptual skills are common. Psychological status is also often altered. Adjustment to disability issues are frequently encountered by people with TBI.

Brain injury can occur in many ways. Traumatic brain injuries typically result from accidents in which the head strikes an object. This is the most common type of traumatic brain injury. Brain injury includes brain shear and diffuse axonal injury. However, other brain injuries, such as those caused by insufficient oxygen, poisoning, or infection, can cause similar deficits.

Mild Traumatic Brain Injury (MTBI) is characterized by one or more of the following symptoms: a brief loss of consciousness, loss of memory immediately before or after the injury, any alteration in mental state at the time of the accident, or focal neurological deficits. In many MTBI cases, the person seems fine on the surface, yet continues to endure chronic functional problems. Some people suffer long-term effects of MTBI, known as postconcussion syndrome (PCS). Persons suffering from PCS can experience significant changes in cognition and personality.

Most traumatic brain injuries result in widespread damage to the brain because the brain ricochets inside the skull during the impact of an accident. Diffuse axonal injury occurs when the nerve cells are torn from one another. Localized damage also occurs when the brain bounces against the skull. The brain stem, frontal lobe, and temporal lobes are particularly vulnerable to this because of their location near bony protrusions.

The brain stem is located at the base of the brain. Aside from regulating basic arousal and regulatory functions, the brain stem is involved in attention and short-term memory. Trauma to this area can lead to disorientation, frustration, and anger. The limbic system, higher up in the brain than the brain stem, helps regulate emotions. Connected to the limbic system are the temporal lobes which are involved in many cognitive skills such as memory and language. Damage to the temporal lobes, or seizures in this area, have been associated with a number of behavioral disorders. The frontal lobe is almost always injured due to its large size and its location near the front of the cranium. The frontal lobe is involved in many cognitive functions and is considered our emotional and personality control center. Damage to this area can result in decreased judgment and increased impulsivity.

The present invention is directed to uses of provides compounds that modulate hepatocyte growth factor/scatter factor (HGF/SF) activity for the treatment, prevention or prophylaxis of diseases and conditions related to demyelinization or trauma to the central nervous system. Included among such compounds is HGF/SF itself and modified HGF/SF molecules that exhibit HGF/SF activity. The aforementioned conditions and diseases are examples of demyelinating diseases and central nervous system trauma amenable to treatment or prophylaxis by the compounds of the invention. The aforementioned diseases and conditions are only meant to be exemplary and non-limiting as to the range of pathologies that are targets for treatment based on the teachings described herein.

Agents and compounds useful for the practice of this invention include those generally set forth above and described specifically herein, and are illustrated in part by the various classes, subgenera and species disclosed herein.

### General Description of HGF/SF Pathway Activators of the Invention

Hepatocyte growth factor (HGF), also known as scatter factor (SF), is a pleiotropic growth factor that stimulates cell growth, cell motility, morphogenesis and angiogenesis. HGF/SF is produced as an inactive monomer (˜100 kDa) which is proteolytically converted to its active form. Active HGF/SF is a heparin-binding heterodimeric protein composed of a 62 kDa a chain and a 34 kDa 0 chain. (Miller, C. M., 1995, Hepatocyte growth factor: a multifunctional cytokine Lancet 345, 293-5). Descriptions of HGF/SF protein may be found in U.S. Pat. Nos. 5,837,676; 5,919,759; 5,965,523; 6,011,009; 6,013,624; 6,498,144; 6,699,837; and 6,303,126, all of which are incorporated herein by reference in their entireties. Included herein are various forms of HGF/SF that retain biological activity for the purposes herein, such as but not limited to fragments, fusion proteins or polypeptides, muteins, truncates, and other modified forms of HGF that have the ability to activate HGF pathways, such as are described in U.S. Pat. No. 6,566,098. Fusion polypeptides comprise molecules wherein HGF protein is expressed in tandem with another protein or polypeptide, for the purpose, for example, of targeted delivery; exemplary guidance therefor may be found in U.S. Pat. No. 6,994,857. Methods for ascertaining HGF/SF agonist activity of such modified HGF/SF molecules useful in the practice of the present invention is readily achieved by following the teachings herein. Such HGF protein and related molecules can be administered directly to a subject in need of such treatment or prophylaxis of disease. The invention is further directed to DNA and other nucleic acid molecules encoding HGF/SF or other proteins with HGF/SF activity, such as described in U.S. Pat. No. 6,248,722, which is incorporated herein by reference in its entirety, as well as DNA and nucleic acids encoding the aforementioned fragments, fusion proteins or polypeptides, muteins, truncates, and other modified forms of HGF, such as described in U.S. Pat. No. 6,566,098. Such nucleic acid molecules can be administered to a subject in need of treatment or prophylaxis by directly administering DNA, or nucleic acid other forms that are readily incorporated into cells of the subject's body and induce expression of the encoded protein, such as but not limited to expression vectors such as a plasmid, virus or yeast. Exemplary small molecule activators of HGF pathways are described below.

In certain embodiments, the uses described herein extend to compounds of the general formula (I):

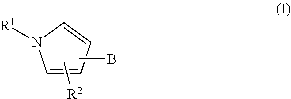

and tautomers and C(5)-positional isomers thereof;
wherein B is a C(3)- or C(5)-substituent selected from the group consisting of -AL¹-A, aryl, heteroaryl and heterocyclic; wherein AL¹ is an optionally substituted C₂₋₆alkenylidene moiety, and A is an optionally substituted alicyclic, heteroalicyclic, aromatic or heteroaromatic moiety;
R¹ is hydrogen, —C(═O)(CH₂)ₘR^{1A}, —C(═O)OR^{1A}, —C(═O)N(R^{1A})₂ or —SO₂R^{1A}; wherein m is an integer from 0-3; each occurrence of R^{1A} is independently hydrogen or an optionally substituted aliphatic, alicyclic, heteroaliphatic, heteroalicyclic, aromatic or heteroaromatic moiety; and
R² is one or more substituents selected from the group consisting of hydrogen, halogen, hydroxyl, —NO₂, —CN, an optionally substituted aliphatic, heteroaliphatic, aromatic, heteroaromatic moiety; —OR^{R}, —S(═O)ₙR^{d}, —NR^{b}R^{c}, and —C(═O)R^{a}; wherein n is 0-2, R^{R} is an optionally substituted aliphatic, heteroaliphatic, aromatic or heteroaromatic moiety;
R^{a}, for each occurrence, is independently selected from the group consisting of hydrogen, hydroxy, aliphatic, heteroaliphatic, aryl and heteroaryl;
R^{b} and R^{c}, for each occurrence, are independently selected from the group consisting of hydrogen; hydroxy; SO₂R^{d}; aliphatic, heteroaliphatic, aryl and heteroaryl;
R^{d}, for each occurrence, is independently selected from the group consisting of hydrogen; —N(R^{e})₂; aliphatic, aryl and heteroaryl; and
R^{e}, for each occurrence, is independently hydrogen or aliphatic.

For example, one class of compounds includes those compounds of formula (I) wherein the nitrogen atom at position 1 is unsubstituted and the compound has the structure (II):

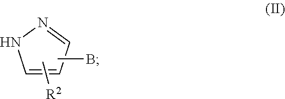

and tautomers thereof;

wherein R² and B are as defined generally above and in classes and subclasses herein.

Another class of compounds includes those compounds of formula (II) having the structure (II^{A}):

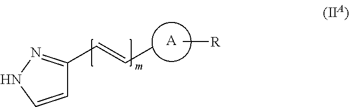

and tautomers thereof;
wherein A is an aromatic or non-aromatic 5-6 membered monocyclic ring, optionally containing 1-4 heteroatoms selected from N, O or S; or an aromatic or non-aromatic 8-12 membered bicyclic ring, optionally containing 1-6 heteroatoms selected from N, O or S; or as defined generally above and in classes and subclasses herein; m is an integer from 0-3; and R is one or two substituents selected from the group consisting of hydrogen, halogen, hydroxyl, —NO₂, —CN, an optionally substituted aliphatic, heteroaliphatic, aromatic, heteroaromatic moiety; —OR^{R}, —S(═O)ₙR^{d}, —NR^{b}R^{c}, and —C(═O)R^{a}; wherein n is 0-2, R^{R} is an optionally substituted aliphatic, heteroaliphatic, aromatic or heteroaromatic moiety;
R^{a}, for each occurrence, is independently selected from the group consisting of hydrogen, hydroxy, aliphatic, heteroaliphatic, aryl and heteroaryl;
R^{b} and R^{c}, for each occurrence, are independently selected from the group consisting of hydrogen; hydroxy; SO₂R^{d}; aliphatic, heteroaliphatic, aryl and heteroaryl;
R^{d}, for each occurrence, is independently selected from the group consisting of hydrogen; —N(R^{e})₂; aliphatic, aryl and heteroaryl; and
R^{e}, for each occurrence, is independently hydrogen or aliphatic.

Another class of compounds includes those compounds of formula (II) having the structure (IIB):

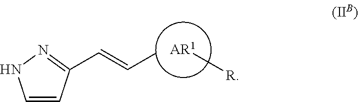

and tautomers thereof;
wherein R is as defined generally above and in classes and subclasses herein; and AR¹ is an optionally substituted aryl moiety.

Another class of compounds includes those compounds of formula (II) having the structure (II^{C}):

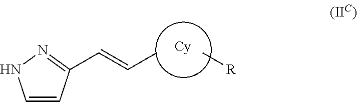

and tautomers thereof;
wherein R is as defined generally above and in classes and subclasses herein; and Cy is an optionally substituted heterocyclic moiety.

Another class of compounds includes those compounds of formula (I) wherein the nitrogen atom at position bears a substituent R¹ and the compound has the structure (III):

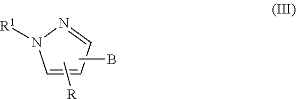

and C(5)-positional isomers thereof;
wherein B is as defined generally above and in classes and subclasses herein; and R¹ is —C(═O)(CH₂)ₘR^{1A}, —C(═O)OR^{1A}, —C(═O)N(R^{1A})₂ or —SO₂R^{1A}; wherein m is an integer from 0-3; and each occurrence of R^{1A} is independently hydrogen or an optionally substituted aliphatic, alicyclic, heteroaliphatic, heteroalicyclic, aromatic or heteroaromatic moiety.

Another class of compounds includes those compounds of formula (III) having the structure (III^{A}):

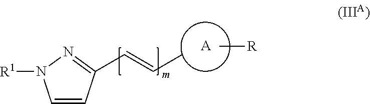

and C(5)-positional isomers thereof;
wherein R¹, R and A are as defined generally above and in classes and subclasses herein; and m is an integer from 0-3.

Another class of compounds includes those compounds of formula (III) having the structure (III^{B}):

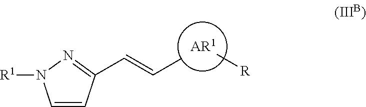

and C(5)-positional isomer thereof;
wherein R and R¹ are as defined generally above and in classes and subclasses herein; and AR¹ is an optionally substituted aryl moiety.

Another class of compounds includes those compounds of formula (III) having the structure (III^{C}):

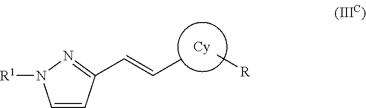

and C(5)-positional isomers thereof;
wherein R and R¹ are as defined generally above and in classes and subclasses herein; and Cy is an optionally substituted heterocyclic moiety.

Another class of compounds includes those compounds of formula (III) having the structure (III^{D}):

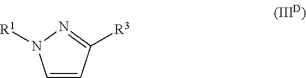

and C(5)-positional isomers thereof;
wherein R¹ is —SO₂R^{1A}; —C(═O)(CH₂)ₘR^{1A}, —C(═O)OR^{1A} or —C(═O)NHR^{1A}, wherein m is an integer from 0-3; and each occurrence of R^{1A} is independently an optionally substituted aliphatic, alicyclic, heteroaliphatic, aryl or heterocyclic moiety; and
R³ is a cis or trans —CH═CH-AR¹, —CH═CH-Cy, phenoxyphenyl, heteroaryl, aryl-substituted heteroaryl, or a heterocyclic group; wherein AR¹ is an optionally substituted aryl or heteroaryl moiety and Cy is an optionally substituted heterocyclic moiety.

A number of important subclasses of each of the foregoing classes useful for the practice of the invention deserve separate mention; these subclasses include subclasses of the foregoing classes in which:

i) R¹ is hydrogen;

ii) R¹ is —C(═O)R^{1A}, —C(═O)NHR^{1A} or —SO₂R^{1A}; wherein each occurrence of R^{1A} is independently alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroaryl or -(heteroalkyl)heteroaryl moiety;

iii) R¹ is —C(═O)R^{1A}, —C(═O)NHR^{1A} or —SO₂R^{1A}; wherein each occurrence of R^{1A} is independently an alkyl, cycloalkyl, heterocyclic or aryl moiety;

iv) R¹ is —SO₂R^{1A}, —C(═O)(CH₂)ₘR^{1A}, —C(═O)OR^{1A} or —C(═O)NHR^{1A}, wherein m is an integer from 0-3; and each occurrence of R^{1A} is independently alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroaryl or -(heteroalkyl)heteroaryl moiety;

v) R¹ is —SO₂R^{1A}, —C(═O)(CH₂)ₘR^{1A}, —C(═O)OR^{1A} or —C(═O)NHR^{1A}, wherein m is an integer from 0-3; and each occurrence of R^{1A} is independently an alkyl, cycloalkyl, heterocyclic or aryl moiety;

vi) R¹ is SO₂AL¹, C(═O)(CH₂)ₘAL¹, C(═O)OAL¹, C(═O)NHAL¹, SO₂Aryl, C(═O)(CH₂)ₘAryl, C(═O)OAryl, C(═O)OHeterocyclic, C(═O)(CH₂)ₘHeterocyclic, or C(═O)NHAryl; wherein m is 0-3; AL¹ is an aliphatic or alicyclic moiety; and AL¹, the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c} or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆alkoxy, aryl, heteroaryl and heterocyclyl; or COCH₂OC₂H₅OCH₃;

vii) compounds of subset yl) above wherein AL¹ is alkyl or cycloalkyl;

viii) R¹ is C(═O)(CH₂)ₘAL¹; C(═O)(CH₂)ₘAryl or C(═O)Heterocyclic; wherein m-1-3; AL¹ is an aliphatic or alicyclic moiety; and AL¹, the aryl and heterocyclic moiety are independently optionally substituted with one or more substituents independently selected from hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃;

ix) compounds of subset vii) above where AL¹ is alkyl or cycloalkyl;

x) R¹ is C(═O)O-AL¹ or C(═O)O-Aryl; wherein AL¹ is an aliphatic or alicyclic moiety; and AL¹ and the aryl moiety are optionally substituted with one or more substituents independently selected from hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆alkoxy, aryl, heteroaryl and heterocyclyl;

xi) compounds of subset x) above where AL¹ is alkyl or cycloalkyl;

xii) R¹ is SO₂AL¹, C(═O)(CH₂)ₘAL¹, C(═O)NHAL¹, SO₂Aryl, C(═O)(CH₂)ₘAryl, C(═O)(CH₂)ₘHeterocyclic or C(═O)NHAryl; wherein m is 0-3; AL¹ is an aliphatic or alicyclic moiety; and AL¹, the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃;

xiii) compounds of subset xii) above where AL¹ is alkyl or cycloalkyl;

xiv) R¹ is C(═O)(CH₂)ₘAL¹ wherein m is 1-3, C(═O)(CH₂)ₘAryl, C(═O)(CH₂)ₘHeterocyclic where m is 0-3; AL¹ is an aliphatic or alicyclic moiety; and AL¹, the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆alkoxy, haloC₁₋₆alkoxy, aryl, heteroaryl and heterocyclyl; or COCH₂OC₂H₅OCH₃;

xv) compounds of subset xiv) above where AL¹ is alkyl or cycloalkyl;

xvi) R¹ as SO₂AL¹, C(═O)AL¹, C(═O)NHAL¹, SO₂Aryl, C(═O)Aryl, or C(═O)NHAryl, wherein AL¹ is an aliphatic or alicyclic moiety; and AL¹ and the aryl moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆alkoxy, aryl, heteroaryl and heterocyclyl; or COCH₂OC₂H₅OCH₃;

xvii) compounds of subset xvi) above wherein AL¹ is alkyl or cycloalkyl;

xviii) R¹ is C(═O)Aryl optionally substituted with one or more substituents independently selected from the group consisting of hydrogen; CN; carboxy ester; —C(═O)R^{a}, or —S(O)ₙR^{d} where n=0-2; C₁₋₆alkoxy substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; —NR^{f}R^{g}; C₁₋₆ alkyl substituted with one or more substituents independently selected from halogen, hydroxy, C₁₋₅ alkoxy, nitro, and N(R^{e})₂, or C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂;

xix) B or R³ is a cis or trans CHCHAryl, CHCHHeterocyclic, phenoxyphenyl, or a heterocyclic group, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂;

xx) B or R³ is a cis or trans CHCHAryl, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂;

xxi) B or R³ is a cis or trans CHCHheterocyclic, phenoxyphenyl, or a heterocyclic group, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂;

xxii) R is one or more substituents selected from the group consisting of hydrogen, halogen, hydroxyl, —NO₂, —CN, alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroaryl or -(heteroalkyl)heteroaryl moiety; hydrogen, alkyl, heteroalkyl, aryl, heteroaryl, -(alkyl)aryl, -(alkyl)heteroaryl, —OR^{R}, —S(═O)ₙR^{R}, —N(R^{R}), —SO₂N(R^{R})₂, —C(═O)R^{R}, —C(═O)N(R^{R})₂, —C(═O)OR^{R}, —N(R^{R})C(═O)R^{R} or —N(R^{R})SO₂R^{R}; wherein n is 0-2, and R^{R}, for each occurrence, is independently hydrogen, lower alkyl, lower heteroalkyl, aryl, heteroaryl, -(alkyl)aryl, or -(alkyl)heteroaryl;

xxiii) R is one or more substituents selected from the group consisting of hydrogen, halogen, hydroxyl, —NO₂, —CN, alkoxy, alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroaryl, -(heteroalkyl)heteroaryl moiety, —S(═O)ₙR^{d}, —NR^{b}R^{c}, and —C(═O)R^{a}; wherein n is 0-2;

xxiv) R is one or more substituents selected from hydrogen; halogen; hydroxy; nitro; CN; aryl; heteroaryl; —C(═O)R^{a}; —NR^{b}R^{c}; —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂;

xxv) R is one or more substituents selected from hydrogen; halogen; hydroxy; nitro; CN; C₁₋₆ alkyl; C₁₋₆ alkoxy; haloC₁₋₆ alkoxy; —C(═O)R^{a}; —C(═O)OR^{a}; —OR^{a} and —NR^{a}R^{b}; wherein R^{a} and R^{b} are independently lower alkyl or any two adjacent R^{a} groups, or R^{a} and R^{b} groups, taken together, may form a heterocyclic moiety;

xxvi) R is one or more substituents selected from hydrogen; halogen; hydroxy or nitro;

xxvii) R^{a}, for each occurrence, is independently selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;

xxviii) R^{b} and R^{c}, for each occurrence, are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂, C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;

xxix) R^{d}, for each occurrence, is independently selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl;

xxx) R^{e}, for each occurrence, is independently hydrogen or C₁₋₆ alkyl;

xxxi) R^{f} and R^{g}, for each occurrence, are independently selected from the group consisting of hydrogen; hydroxy; SO₂R^{d}; C₁₋₆ alkyl substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;

xxxii) R² is one or more substituents selected from the group consisting of hydrogen, halogen, hydroxyl, —NO₂, —CN, alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroaryl or -(heteroalkyl)heteroaryl moiety; hydrogen, alkyl, heteroalkyl, aryl, heteroaryl, -(alkyl)aryl, -(alkyl)heteroaryl, —OR^{R}, —S(═O)ₙR^{R}, —N(R^{R}), —SO₂N(R^{R})₂, —C(═O)R^{R}, —C(═O)N(R^{R})₂, —C(═O)OR^{R}, —N(R^{R})C(═O)R^{R} or —N(R^{R})SO₂R^{R}; wherein n is 0-2, and R^{R}, for each occurrence, is independently hydrogen, lower alkyl, lower heteroalkyl, aryl, heteroaryl, -(alkyl)aryl, or -(alkyl)heteroaryl;

xxxiii) R² is one or more substituents selected from the group consisting of hydrogen, halogen, hydroxyl, —NO₂, —CN, alkoxy, alkyl, alkenyl, alkynyl, cycloalkyl, cycloalkenyl, cycloalkynyl, heterocyclic, aryl, heteroaryl, -(alkyl)aryl, -(heteroalkyl)aryl, -(alkyl)heteroary, -(heteroalkyl)heteroaryl moiety, —S(═O)ₙR^{d}, —NR^{b}R^{c}, and —C(═O)R^{a}; wherein n is 0-2;

xxxiv) R² is one or more substituents selected from hydrogen; halogen; hydroxy; nitro; CN; aryl; heteroaryl; —C(═O)R^{a}; —NR^{b}R^{e}; —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂;

xxxv) R² is one or more substituents selected from hydrogen; halogen; hydroxy; nitro; CN; C₁₋₆ alkyl; C₁₋₆ alkoxy; haloC₁₋₆ alkoxy; —C(═O)R^{a}; —C(═O)OR^{a}; —OR^{a} and —NR^{a}R^{b}; wherein R^{a} and R^{b} are independently lower alkyl or any two adjacent R^{a} groups, or R^{a} and R^{b} groups, taken together, may form a heterocyclic moiety;

xxxvi) A is an alicyclic, heteroalicyclic, aromatic or heteroaromatic moiety;

xxxvii) A is an optionally substituted aromatic or non-aromatic 5-6 membered monocyclic ring, optionally containing 1-4 heteroatoms selected from N, O or S; or an optionally substituted aromatic or non-aromatic 8-12 membered bicyclic ring, optionally containing 1-6 heteroatoms selected from N, O or S;

xxxviii) A is an aromatic or non-aromatic 5-6 membered monocyclic ring or 8-12 membered bicyclic ring, optionally substituted with one or more substituents selected from hydrogen; halogen; hydroxy; nitro; CN; C₁₋₆ alkyl; C₁₋₆ alkoxy; haloC₁₋₆ alkoxy; —C(═O)R^{a}; —C(═O)OR^{a}; —OR^{a} and —NR^{a}R^{b}; wherein R^{a} and R^{ip} are independently lower alkyl or any two adjacent R^{a} groups, or R^{a} and R^{b} groups, taken together, may form a heterocyclic moiety;

xxxix) A is an aromatic or non-aromatic 5-6 membered monocyclic ring or 8-12 membered bicyclic ring, optionally substituted with one or more substituents selected from hydrogen; Cl; hydroxy; nitro; CN; —OCF₃; —C(═O)OMe; —C(═O)Me; —OMe; methyldioxyl; —NMe₂ and morpholinyl;

xl) A is optionally substituted aryl;

xli) A is optionally substituted phenyl or naphthyl;

xlii) A is optionally substituted heteroaryl;

xliii) A has the structure:

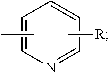

wherein R represents one or more substituents, as defined in subsets xxii)-xxvii);

xlv) A is an optionally substituted C₁₋₆cycloalkyl or C₁₋₆cycloalkenyl moiety;

xlvi) A is optionally substituted cyclohexenyl;

xlvii) A is an optionally substituted heterocyclic moiety;

xlviii) A and/or Cy is one of:

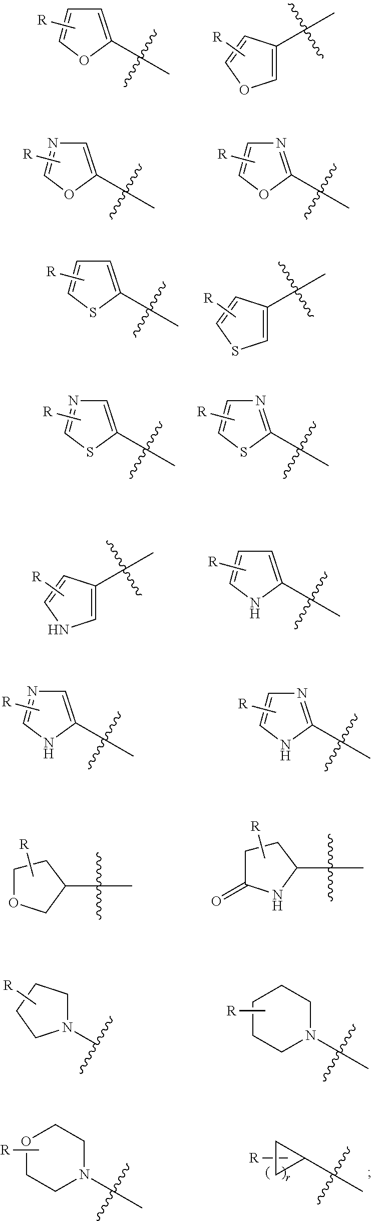

wherein R represents one or more substituents, as defined in subsets xxii)-xxvii); and r is an integer from 1-6;

xlix) A and/or Cy is an optionally substituted 5-membered heterocyclic moiety having the structure:

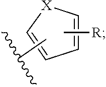

wherein R represents one or more substituents, as defined in subsets xxii)-xxvii); and X is O, S or NR^{N}; wherein R^{N} is hydrogen, lower alkyl, aryl, acyl or a nitrogen protecting group;

l) A and/or Cy is an optionally substituted 5-membered heterocyclic moiety having the structure:

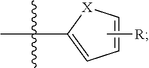

wherein R represents one or more substituents, as defined in subsets xxii)-xxvii); and X is O, S or NR^{N}; wherein R^{N} is hydrogen, lower alkyl, aryl, acyl or a nitrogen protecting group;

li) B is a moiety having the structure:

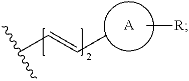

wherein A and R are as defined in classes and subclasses herein;

lii) B is a moiety having one of the structures:

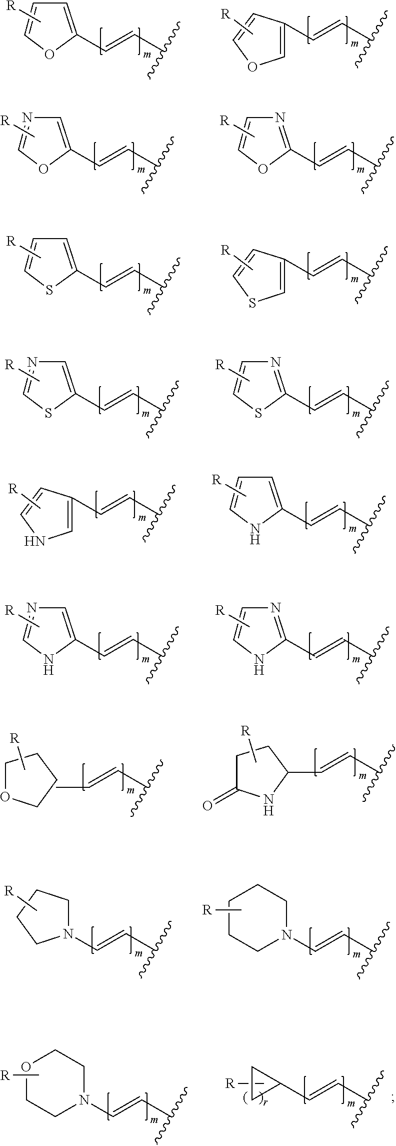

wherein R represents one or more substituents, as defined in subsets xxii)-xxvii); m is an integer from 1-3; and r is an integer from 1-6;

liii) AR is phenyl or naphthyl; and/or

liv) AR¹ is phenyl or naphthyl.

It will be appreciated that for each of the classes and subclasses described above and herein, any one or more occurrences of aliphatic and/or heteroaliphatic may independently be substituted or unsubstituted, linear or branched, saturated or unsaturated; any one or more occurrences of alicyclic and/or heteroalicyclic may independently be substituted or unsubstituted, saturated or unsaturated; and any one or more occurrences of aryl and/or heteroaryl may independently be substituted or unsubstituted.

The reader will also appreciate that all possible combinations of the variables described in i) through liv) above (e.g., R, R¹, and B, among others) are considered compounds useful for the practice of the invention. Thus, the method of the invention encompass any and all compounds of formula I generated by taking any possible permutation of variables R, R¹, and B, and other variables/substituents (e.g., A, R^{1A}, etc.) as further defined for R, R¹, and B, described in i) through liv) above.

For example, an exemplary combination of variables described in i) through liv) above includes uses of those compounds of Formula I wherein:
B is a C(3)- or C(5)-substituent selected from the group consisting of optionally substituted cis or trans CHCHAryl, CHCHHeterocyclic, phenoxyphenyl and a heterocyclic group;
R¹ is C(═O)Aryl optionally substituted with one or more substituents independently selected from the group consisting of hydrogen; CN; carboxy ester; —C(═O)R^{a}, or —S(O)ₙR^{d} where n=0-2; C₁₋₆alkoxy substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; —NR^{f}R^{g}; C₁₋₆ alkyl substituted with one or more substituents independently selected from halogen, hydroxy, C₁₋₅ alkoxy, nitro, and N(R^{e})₂, or C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆alkoxy, aryl, heteroaryl and heterocyclyl; and
R is one or more substituents selected from hydrogen; halogen; hydroxy; nitro; CN; aryl; heteroaryl; —C(═O)R^{a}; —NR^{b}R^{c}; —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂;
wherein R^{a}, for each occurrence, is independently selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c}, for each occurrence, are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d}, for each occurrence, is independently selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
R^{e}, for each occurrence, is independently hydrogen or C₁₋₆ alkyl.

Other exemplary combinations of substituents in compounds useful for the purposes described herein are illustrated by compounds of the following subgroups I-XIII:

I. Compounds Having the Structure:

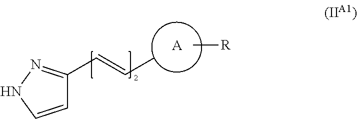

tautomers thereof; and pharmaceutically acceptable derivatives thereof;
wherein A and R are as defined generally and in classes and subclasses herein. In certain embodiments, A represents an optionally substituted aromatic or non-aromatic 5-6 membered monocyclic ring, optionally containing 1-4 heteroatoms selected from N, O or S; or an optionally substituted aromatic or non-aromatic 8-12 membered bicyclic ring, optionally containing 1-6 heteroatoms selected from N, O or S. In certain other embodiments, R is one or more substituents selected from the group consisting of hydrogen; halogen; hydroxy; nitro; CN; aryl; heteroaryl; —C(═O)R^{a}; —NR^{b}R^{c}; —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl;
wherein each occurrence of R^{a} is independently selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
each occurrence of R^{b} and R^{c} is independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
each occurrence of R^{d} is independently selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
each occurrence of R^{e} is independently hydrogen or C₁₋₆ alkyl.

A non-limiting example of compounds of this subgroup includes:

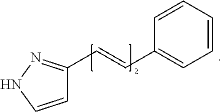

II. Compounds Having the Structure:

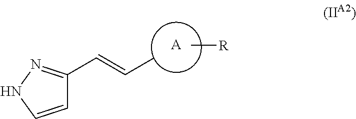

tautomers thereof, and pharmaceutically acceptable derivatives thereof;
wherein A and R are as defined generally and in classes and subclasses herein.

In certain exemplary embodiments, A is an aromatic or non-aromatic 5-6 membered monocyclic ring, optionally containing 1-4 heteroatoms selected from N, O or S; or an aromatic or non-aromatic 8-12 membered bicyclic ring, optionally containing 1-6 heteroatoms selected from N, O or S;

and R is one or more substituents selected from the group consisting of hydrogen; halogen; hydroxy; nitro; CN; aryl; heteroaryl; —C(═O)R^{a}; —NR^{b}R^{c}; —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂;
wherein each occurrence of R^{a} is independently selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
each occurrence of R^{b} and R^{c} is independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
each occurrence of R^{d} is independently selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
each occurrence of R^{e} is independently hydrogen or C₁₋₆ alkyl;
or a prodrug, salt, hydrate, or ester thereof.

III. Compounds Having the Structure:

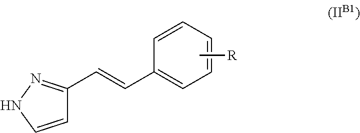

tautomers thereof, and pharmaceutically acceptable derivatives thereof;
wherein R is as defined generally and in classes and subclasses herein. In certain embodiments, R is as defined for subgroup II above.

Non-limiting examples of compounds this subgroup include:

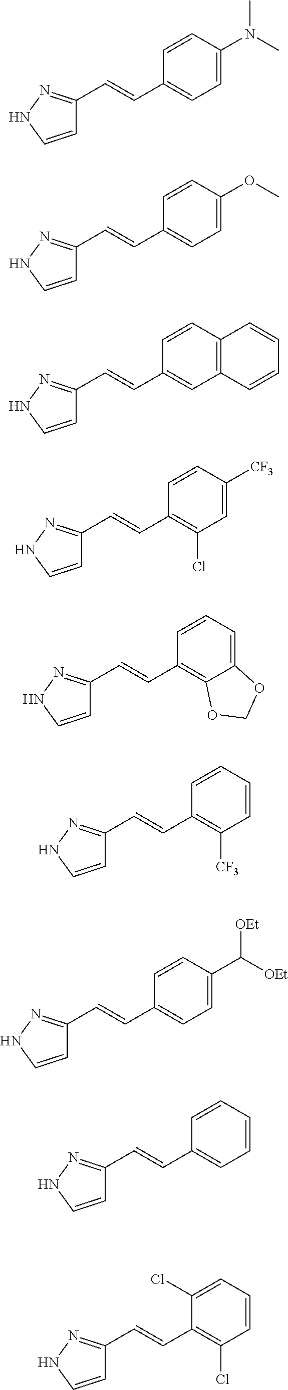

IV. Compounds Having the Structure:

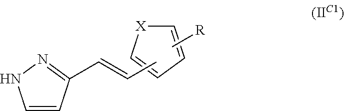

tautomers thereof, and pharmaceutically acceptable derivatives thereof;
wherein R is as defined generally and in classes and subclasses herein; and X is O, S or NR^{N} wherein R^{N} is hydrogen, alkyl, heteroalkyl, aryl, heteroaryl, -(alkyl)aryl, -(alkyl)heteroaryl, acyl or a nitrogen protecting group. In certain embodiments, R is as defined for subgroup II above.

Non-limiting examples of compounds of this subgroup include:

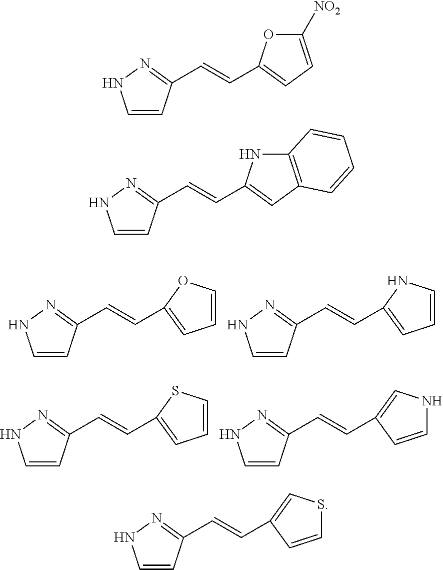

V. Compounds Having the Structure:

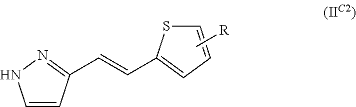

tautomers thereof, and pharmaceutically acceptable derivatives thereof;
wherein R is as defined generally and in classes and subclasses herein. In certain embodiments, R is as defined in subgroup II above.

VI. Compounds Having the Structure:

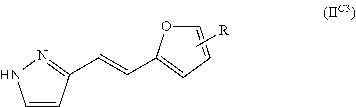

tautomers thereof, and pharmaceutically acceptable derivatives thereof;
wherein R is as defined generally and in classes and subclasses herein. In certain embodiments, R is as defined in subgroup II above.

VII. Compounds Having the Structure:

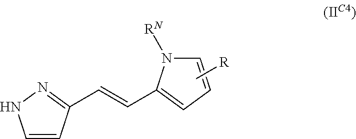

tautomers thereof, and pharmaceutically acceptable derivatives thereof;
wherein R is as defined generally and in classes and subclasses herein; and R^{N} is hydrogen, alkyl, heteroalkyl, aryl, heteroaryl, -(alkyl)aryl, -(alkyl)heteroaryl, acyl or a nitrogen protecting group. In certain embodiments, R is as defined in subgroup II above. In certain other embodiments, R^{N} is hydrogen.

In another broad aspect of the present invention, the following disubstituted compounds and their C(5)-positional isomers are embraced for the methods described herein, such compounds exhibiting HGF/SF mimicking/modulating activity, and in particularly activity similar to that of HGF/SF.

VIII. Compounds Having the Structure:

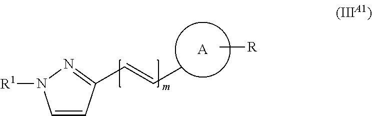

C(5)-positional isomers thereof; and pharmaceutically acceptable derivatives thereof;
wherein R¹ and R are as defined generally and in classes and subclasses herein; m is an integer from 0-3; and A represents an optionally substituted aromatic or non-aromatic 5-6 membered monocyclic ring, optionally containing 1-4 heteroatoms selected from N, O or S; or an optionally substituted aromatic or non-aromatic 8-12 membered bicyclic ring, optionally containing 1-6 heteroatoms selected from N, O or S. In certain other embodiments, R¹ is SO₂AL², C(═O)(CH₂)ₘAL², C(═O)OAL², C(═O)NHAL², SO₂Aryl, C(═O)(CH₂)ₘAryl, C(═O)OAryl, C(═O)Oheterocyclic, C(═O)(CH₂)ₘHeterocyclic, or C(═O)NHAryl; wherein AL² is an alkyl or cycloalykl moiety; and AL², the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃. In certain embodiments, R is as defined in subgroup II above.

IX. Compounds Having the Structure:

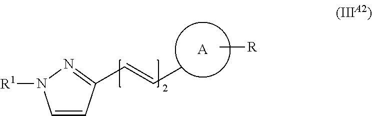

C(5)-positional isomers thereof; and pharmaceutically acceptable derivatives thereof;
wherein A, R¹ and R are as defined generally and in classes and subclasses herein. In certain embodiments, A represents an optionally substituted aromatic or non-aromatic 5-6 membered monocyclic ring, optionally containing 1-4 heteroatoms selected from N, O or S; or an optionally substituted aromatic or non-aromatic 8-12 membered bicyclic ring, optionally containing 1-6 heteroatoms selected from N, O or S. In certain other embodiments, R¹ is SO₂AL², C(═O)(CH₂)ₘAL², C(═O)OAL², C(═O)NHAL², SO₂Aryl, C(═O)(CH₂)ₘAryl, C(═O)OAryl, C(═O)Oheterocyclic, C(═O)(CH₂)ₘHeterocyclic, or C(═O)NHAryl; wherein m is an integer from 0-3; AL² is an alkyl or cycloalykl moiety; and AL², the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃. In certain embodiments, R is as defined in subgroup II above.

X. Compounds Having the Structure:

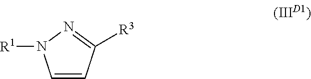

C(5)-positional isomers thereof; and pharmaceutically acceptable derivatives thereof;
wherein R¹ is C(═O)(CH₂)ₘAL², C(═O)OAL², C(═O)(CH₂)ₘAryl, C(═O)OAryl, C(═O)Heteroaryl or C(═O)Heterocyclic; where m is an integer from 1-3; AL² is an aliphatic or alicyclic moiety; and AL², the aryl, heteroaryl and heterocyclic moiety are independently optionally substituted with one or more substituents independently selected from hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃; and
R³ is a cis or trans CHCHAryl, CHCHHeterocyclic, phenoxyphenyl, or a heterocyclic group, wherein the aryl, heterocyclic or phenoxyphenyl moiety may be optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl;
wherein R^{a} is selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl;
and R^{e} is hydrogen or C₁₋₆ alkyl.

In certain embodiments, for the compounds of formula (III^{D1}) above, AL² is an alkyl or cycloalkyl moiety.

In certain embodiments, for the compounds of formula (III^{D1}) above, R³ is a cis or trans CHCHHeterocyclic, phenoxyphenyl, or a heterocyclic group, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl;

wherein R^{a} is selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
R^{e} is hydrogen or C₁₋₆ alkyl.

Non-limiting examples of compounds of this subgroup include:

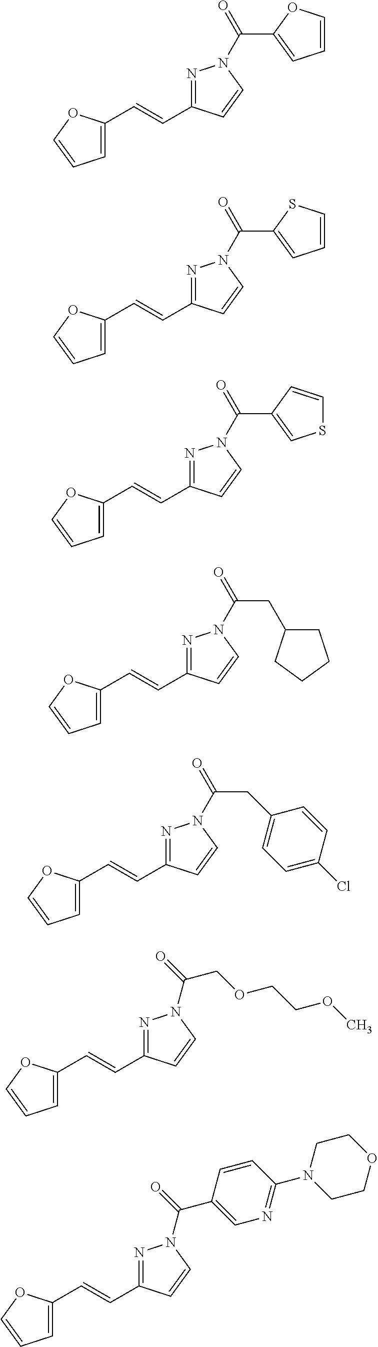

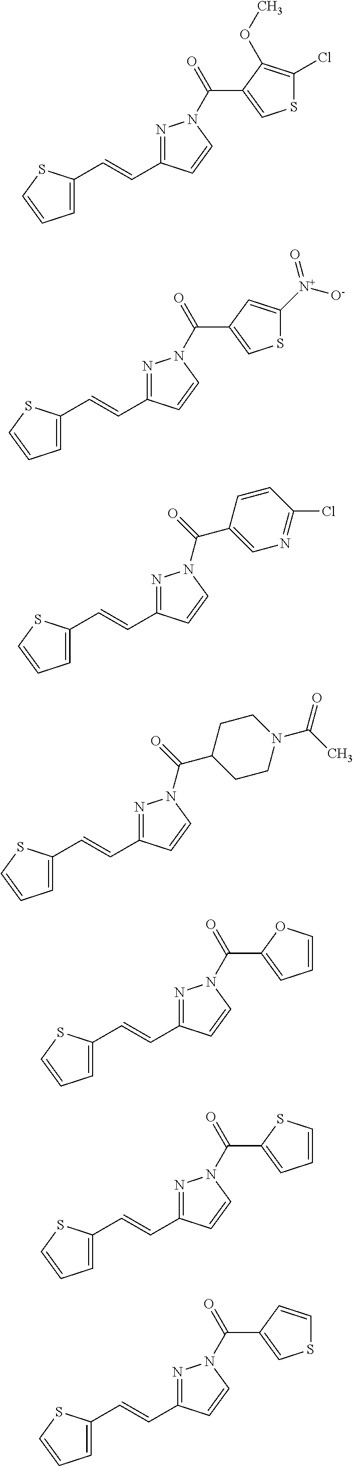

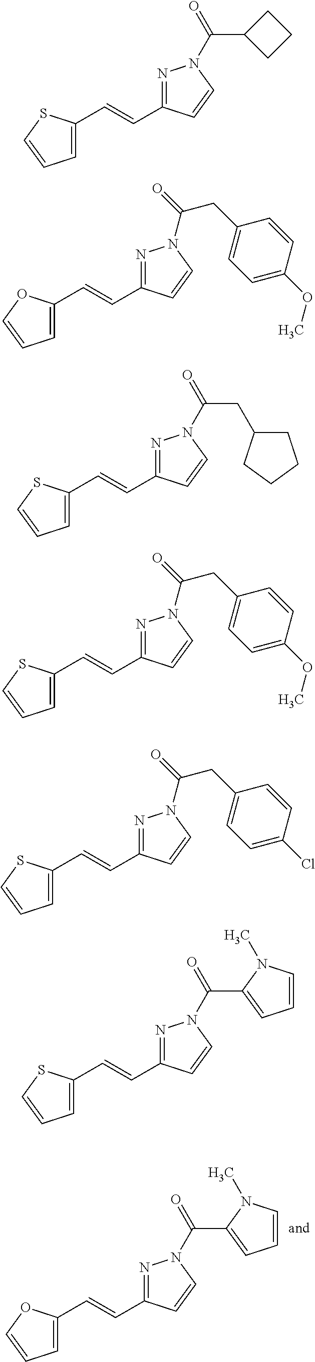

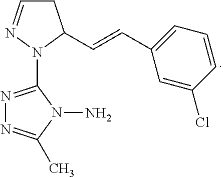

As mentioned above and herein throughout, although the compound structures depicted herein are substituted at the 1 and 3 positions, the invention embraces such positional isomers where the 3-substituent is at the 5 position, and any combination thereof.

In another aspect of compounds of Formula (III^{D1}), R³ is a cis or trans CHCHAryl, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆alkoxy, aryl, heteroaryl and heterocyclyl;

wherein R^{a} is selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
R^{e} is hydrogen or C₁₋₆ alkyl.

Non-limiting examples of compounds of this subgroup include:

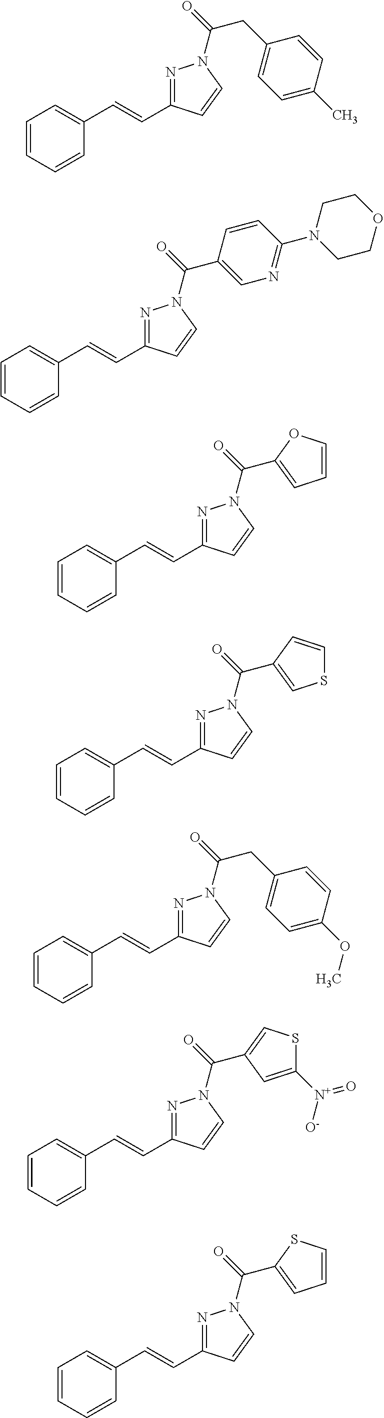

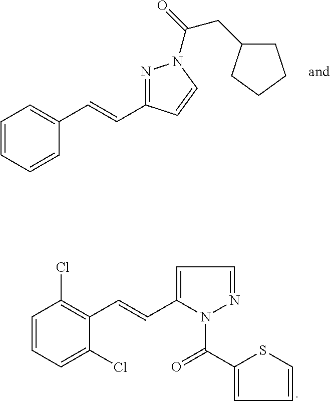

XI. Compounds Having the Structure:

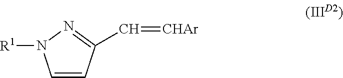

C(5)-positional isomers thereof; and pharmaceutically acceptable derivatives thereof;
wherein R¹ is SO₂AL², C(═O)(CH₂)ₘAL², C(═O)OAL², C(═O)NHAL², SO₂Aryl, C(═O)(CH₂)ₘAryl, C(═O)OAryl, C(═O)Oheterocyclic, C(═O)(CH₂)ₘHeterocyclic, or C(═O)NHAryl; wherein m is an integer from 1-3; AL² is an aliphatic or alicyclic moiety; and AL², the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆alkoxy, aryl, heteroaryl and heterocyclyl; or COCH₂OC₂H₅OCH₃; and
CH═CHAr is a cis or trans CH═CHAryl optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅alkoxy, nitro, and N(R^{e})₂;
wherein R^{a} is selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
R^{e} is hydrogen or C₁₋₆ alkyl.

In certain embodiments, for compounds of Formula (III^{D2}), R¹ is C(═O)(CH₂)ₘAL², C(═O)OAL², C(═O)(CH₂)ₘAryl, C(═O)OAryl, C(═O)OHeterocyclic or C(═O)(CH₂)ₘHeterocyclic; wherein m is an integer from 1-3; AL² is an aliphatic or alicyclic moiety; and AL², the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃.

Non-limiting examples of compound of this subgroup include:

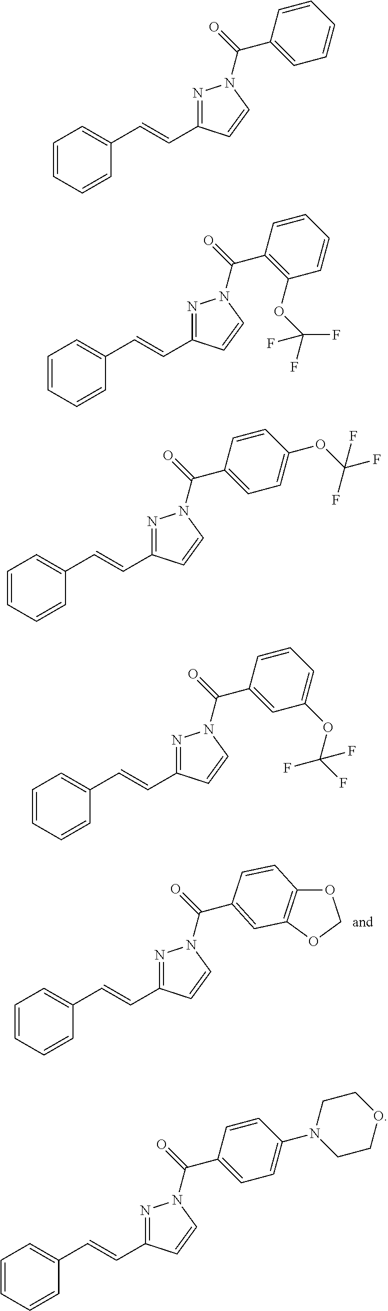

In certain other embodiments, for compounds of Formula (III^{D2}), R¹ is SO₂AL², C(═O)AL², C(═O)NHAL², SO₂Aryl, C(═O)Aryl, or C(═O)NHAryl; wherein AL² is an aliphatic or alicyclic moiety; and AL² and the aryl moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃.

Non-limiting examples of this subgroup include:

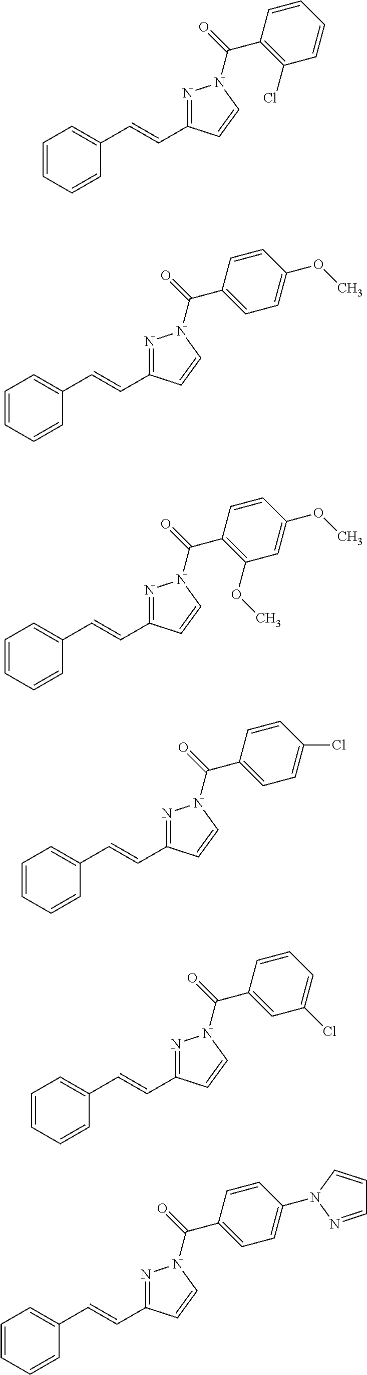

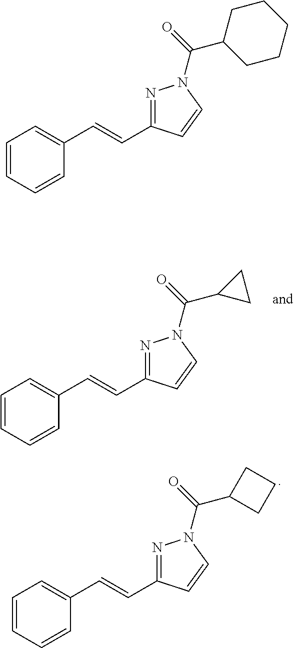

In certain embodiments, for the compounds of subgroup XI above, AL² is an alkyl or cycloalkyl moiety.

XII. Compounds Having the Structure:

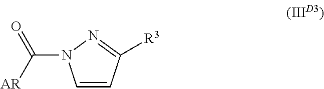

C(5)-positional isomer thereof; and pharmaceutically acceptable derivatives thereof;

wherein AR is an optionally fused 3-12 membered aromatic or alicyclic mono- or bicyclic-ring containing 0-3 heteroatoms selected from the group consisting of N, O, and S optionally substituted with one or more substituents independently selected from the group consisting of hydrogen; halogen; hydroxy; nitro; CN; aryl; heteroaryl; heterocycle; carboxy ester; —C(═O)R^{a}, —NR^{b}R^{c}, or —S(O)ₙR^{d} where n=0-2; C₁₋₆alkoxy substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; —NR^{f}R^{g}; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆alkoxy, aryl, heteroaryl and heterocyclyl; and

R³ is a cis or trans CHCHheterocyclic, phenoxyphenyl, or a heterocyclic group, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl;
wherein R^{a} is selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl;
R^{e} is hydrogen or C₁₋₆ alkyl; and
R^{f} and R^{g} are independently selected from the group consisting of hydrogen; hydroxy; SO₂R^{d}; C₁₋₆ alkyl substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂.

In certain embodiments, when AR is aryl substituted with C₁₋₆alkyl, the C₁₋₆alkyl moiety is substituted. In certain exemplary embodiments, the substituents are independently selected from halogen, hydroxy, C₁₋₅alkoxy, nitro and N(R^{e})₂.

Non-limiting examples of compounds of this subgroup include:

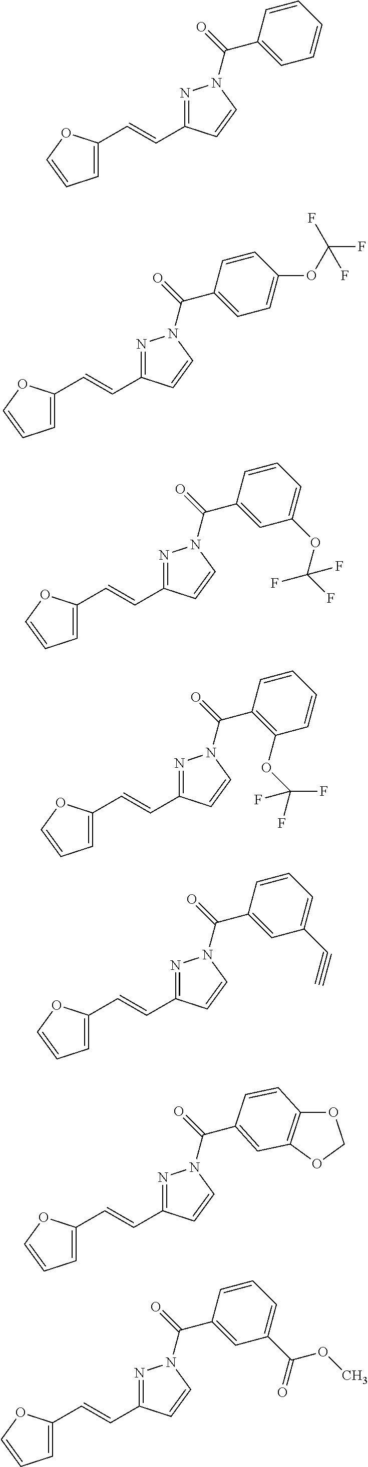

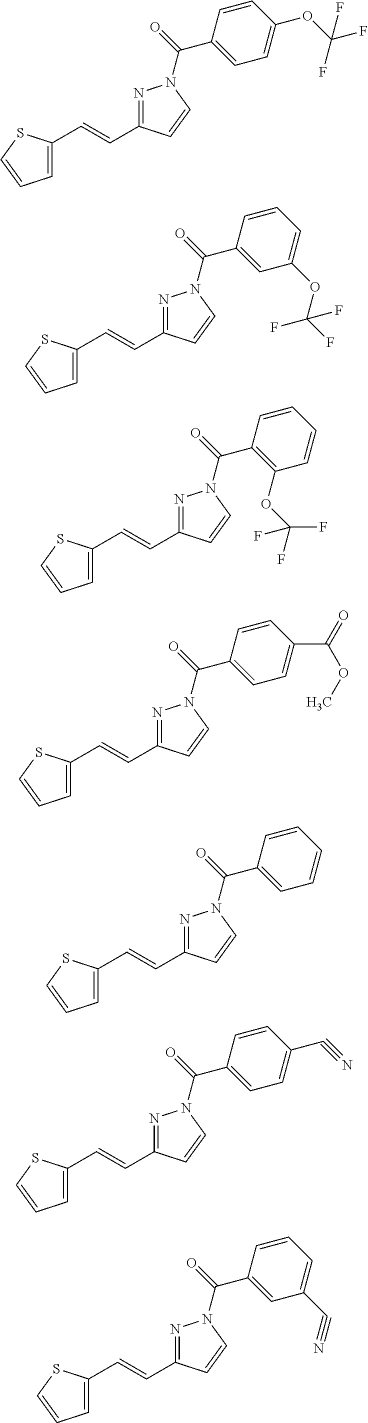

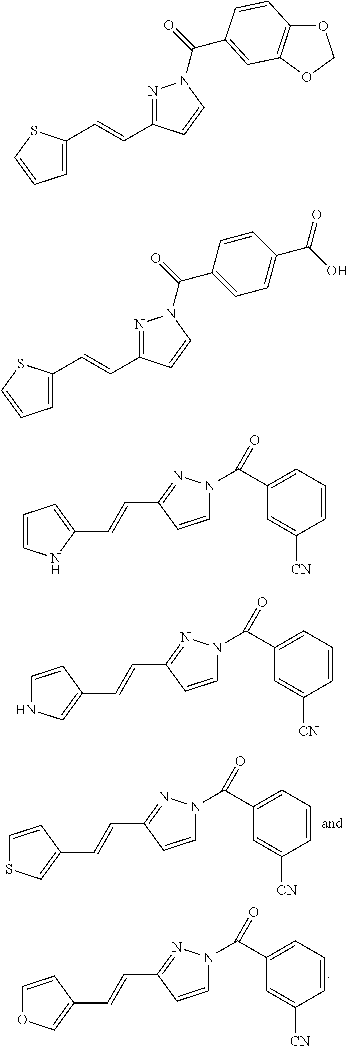

XIII. Compounds Having the Structure:

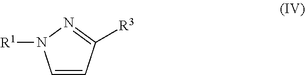

C(5)-positional isomers thereof; and pharmaceutically acceptable derivatives thereof;
wherein R¹ is S(═O)₂Alkyl, S(═O)₂Aryl, C(═O)Alkyl, C(═O)Aryl or C(═O)NHAlkyl or C(═O)NHAryl;
R³ is a cis or trans CHCHAryl, CHCHHeterocyclic, phenoxyphenyl, or a heterocyclic group. With regard to the compounds of Formula IV herein, the term “alkyl” means straight-chain, branched-chain or cyclo saturated aliphatic hydrocarbon groups preferably containing from one to about 6 carbon atoms. Representative of such straight-chain groups are methyl, ethyl, butyl, pentyl, hexyl and the like. Examples of branched-chain groups include isopropyl, isobutyl and t-butyl. Cycloalkyl includes groups such as but not limited to cyclopropyl, cyclobutyl, cyclopentyl and cyclohexyl. The term “aryl” refers to, for example, phenyl, biphenyl and naphthyl groups, which are optionally substituted by one or more halogen (F, Cl, Br and I), C1 to C4 alkyl, or C1 to C4 alkyloxy, where alkyloxy refers to an alkyl group as defined above attached to the remainder of the molecule by oxygen. Examples of alkyloxy include methoxy, ethoxy, propoxy, isopropoxy and the like. The term “heteroaryl” refers to heterocyclic groups containing 4-10 ring members and 1-3 heteroatoms selected from the group consisting of oxygen, nitrogen and sulfur. Examples include but are not limited to isoxazolyl, phenylisoxazolyl, furyl, pyrimidinyl, quinolyl, tetrahydroquinolyl, pyridyl, imidazolyl, pyrrolidinyl, 1,2,4-triazoylyl, thiazolyl, thienyl, and the like. The aryl or heteroaryl group may be optionally substituted by one or more halogen (F, Cl, Br and I), C1 to C4 alkyl, C1 to C4 alkyloxy as described above, trifluoromethyl, difluoromethyl, nitro, hydroxy, amine (optionally alkyl substituted), or another aryl or another heteroaryl group as described above.

Non-limiting examples of compounds of formula IV useful in the practice of the invention include:
(4-chlorophenyl)[5-(2-(2-thienyl)vinyl)-1H-pyrazol-1-yl]methanone;
1-(methylsulfonyl)-5-(2-(2-thienyl)vinyl)-1H-pyrazole;
2,2-dimethyl-1-(5-(2-(2-thienyl)vinyl)-1H-pyrazole-1-yl)propan-1-one
N-methyl-5-(2-(2-thienyl)vinyl)-1H-pyrazole-1-carboxamide
(4-chlorophenyl)(5-(3-phenylisoxazol-5-yl)-1H-pyrazol-1-yl)methanone
(4-chlorophenyl)(5-(3-(4-chlorophenyl)-5-methylisoxazol-4-yl)-1H-pyrazol-1-yl)methanone
(4-chlorophenyl)(5-(5-(2-thienyl)-2-thienyl)-1H-pyrazol-1-yl)methanone
(2,4-dichlorophenyl)(5-(5-(2,4-difluorophenyl)-2-furyl)-1H-pyrazol-1-yl)methanone
N1-phenyl-5-(2-(2-thienyl)vinyl)-1H-pyrazole-1-carboxamide
(4-chlorophenyl)(5-(2-(5-(2-thienyl)-2-thienyl)-4-methyl-1,3-thiazol-5-yl)-1H-pyrazol-1-yl)methanone
(5-benzhydryl-1H-pyrazol-1-yl)(4-chlorophenyl)methanone
N1-(4-chlorophenyl)-5-(2-(2-thienyl)vinyl)-1H-pyrazole-1-carboxamide
(4-chlorophenyl)(5-(2-methylimidazo(1,2-a)pyridin-3-yl)-1H-pyrazol-1-yl)methanone
2-chloro-6-(4-(1-(4-chlorobenzyl)-1H-pyrazol-5-yl)phenoxy)benzonitrile; and
1-((4-chlorophenyl)sulfonyl)-5-(2-(2-thienyl)vinyl)-1H-pyrazole.

Other compounds of formula IV useful for the practice of the present invention include:

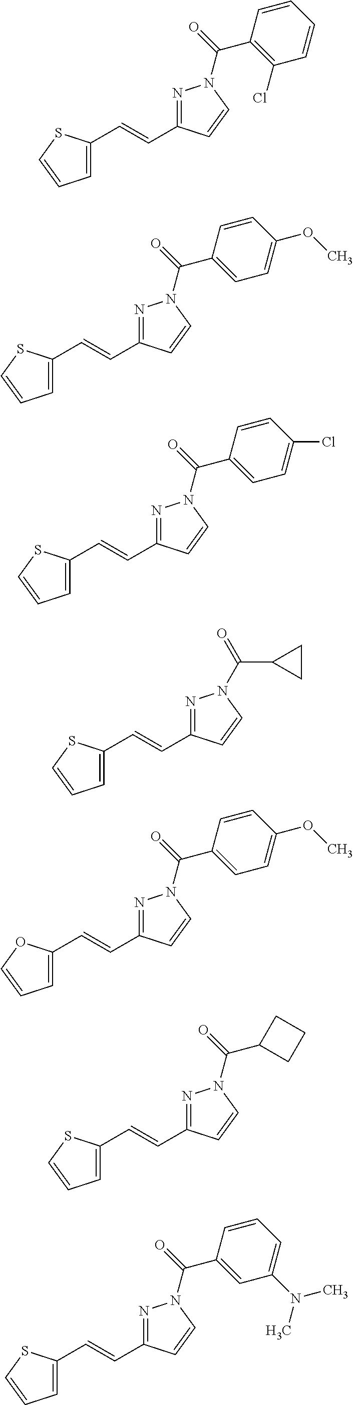

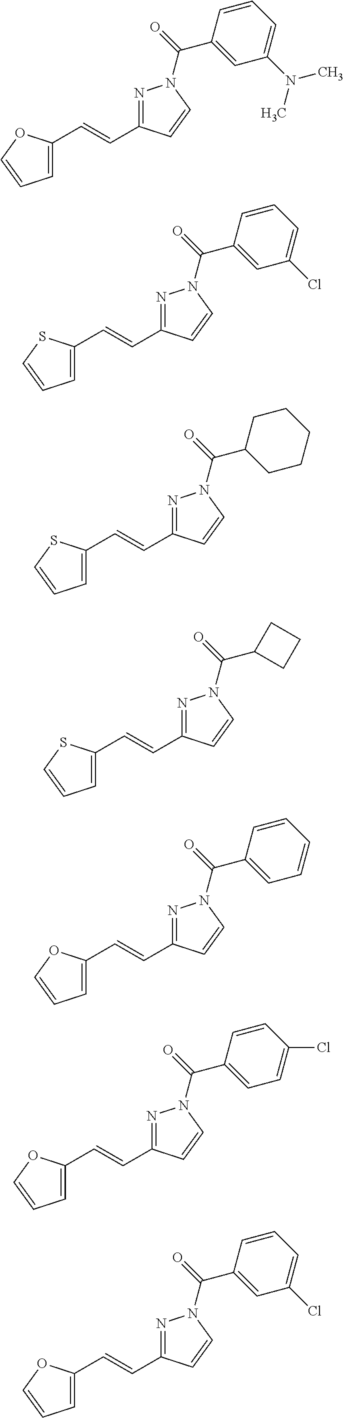

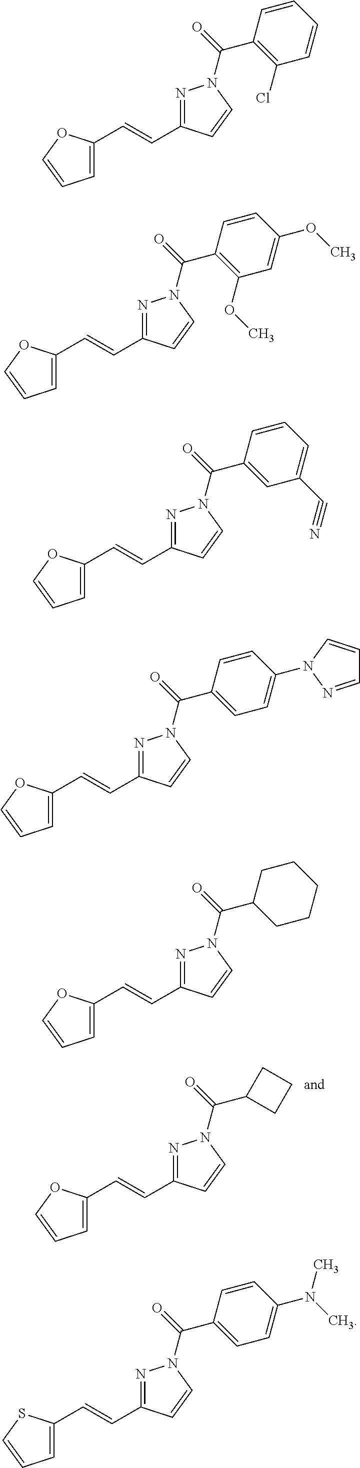

It will be appreciated that each of the compounds described herein and each of the subclasses of compounds described above (I-XIII) may be substituted as described generally herein, or may be substituted according to any one or more of the subclasses described above and herein [e.g., i)-liv)].

In another embodiment, the invention is directed to the use for any one or more of the aforementioned purposes of compounds that activate HGF/SF pathways with the general formula V:

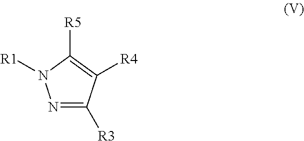

wherein R3 and R5 are independently or together a straight-chain or branched C1-C6 alkyl optionally substituted with a cyano or halogen, halogen, trifluoromethyl or difluoromethyl groups;
R1 is hydrogen, methyl, CO-Aryl, SO₂-Aryl, CO-heteroaryl, or CO-alkyl; and
R4 is CH₂-Aryl, halogen, arylcarbonylvinyl or S-heteroaryl.

R3 and R5 preferably may be methyl, t-butyl or chloro groups. The aryl group of substituent R1 is preferably an aromatic group such as phenyl, naphthyl, or biphenyl, substituted with one or more halogen, C1 to C4 alkyl or C1 to C4 alkyloxy groups. The heteroaryl group of substituent R1 preferably is a 3-aryl-substituted isoxazole or 3-aryl-substituted thienyl group. The alkyl group of substituent R1 preferably is t-butyl, or a C1-C6 straight, branched or cycloalkyl group. In a most preferred embodiment, R3 is methyl, R5 is chloro, R1 is methyl, and R4 is 4-chlorophenylcarbonylvinyl group.

Non-limiting example of modulators of HGF/SF activity of Formula V useful for the purposes described herein include the following compounds:
3-(5-chloro-1,3-dimethyl-1H-pyrazol-4-yl)-1-(4-chlorophenyl)prop-2-en-1-one
[4-(2,6-dichlorobenzyl)-3,5-dimethyl-1H-pyrazol-1-yl][3-(2,6-dichlorophenyl)-5-methylisoxazol-4-yl]methanone
(4-(2-chloro-6-fluorobenzyl)-3,5-dimethyl-1H-pyrazole-1-yl)(3-(2,6-dichlorophenyl)-5-methylisoxazol-4-yl)methanone
4-(2-chloro-6-fluorobenzyl)-1-((3,4-dichlorophenyl)sulfonyl)-3,5-dimethyl-1H-pyrazole
4-(2-chloro-6-fluorobenzyl)-1,3,5-trimethyl-1H-pyrazole
4-(2-chloro-6-fluorobenzyl)-3,5-dimethyl-1H-pyrazole
(4-bromo-3,5-dimethyl-1H-pyrazol-1-yl)(3-(2,6-dichlorophenyl)isoxazole-4-carbohydrazide)
3-(4-(2,6-dichlorobenzyl)-3,5-dimethyl-1H-pyrazol-1-yl)propanenitrile
3,5-di(tert-butyl)-4-(2-chloro-6-fluorobenzyl)-1H-pyrazole
(4-(2-chloro-6-fluorobenzyl)-3,5-dimethyl-1H-pyrazole-1-yl)(2,6-dichlorophenyl)methanone
1-(4-(2-chloro-6-fluorobenzyl)-3,5-dimethyl-1H-pyrazole-1-yl)2,2-dimethylpropan-1-one
(4-(2-chloro-6-fluorobenzyl)-3,5-dimethyl-1H-pyrazole-1-yl)(4-chlorophenyl)methanone
(4-(2-chloro-6-fluorobenzyl)-3,5-dimethyl-1H-pyrazole-1-yl)(2-thienyl)methanone; and
(4-chlorophenyl)(3,5-dimethyl-4-((1-methyl-1H-imidazol-2-yl)thio)-1H-pyrazol-1-yl)methanone.

In a further embodiment, the invention is directed to the use for any one or more of the aforementioned purposes of compounds that activate HGF/SF pathways with the general formula VI:

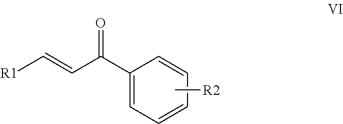

wherein R1 is Aryl or Heteroaryl; and
R2 is one or more halogen, nitro, C1 to C4 straight-chained alkyl, branched alkyl, or cycloalkyl, or C1 to C4 alkyloxy groups.

The definitions of the aforementioned substituents are described hereinabove. Preferably, R1 is a phenyl group substituted with one or more halogen, C1 to C4 alkyl, or C1 to C4 alkyloxy groups, or a heteroaryl, most preferably 4-bromo-2-thienyl, 4-pyridyl, 2-furyl, 3-thienyl, substituted with halogens and/or C1 to C4 alkyl. R2 preferably is halogen (F, Cl, Br), nitro, or a C1 to C4 straight-chained alkyl, branched alkyl, or cycloalkyl group or a C1 to C4 alkyloxy group; most preferably, R2 is a methyl group and a chloro group.

Non-limiting examples of modulators of Formula VI include:
1-(4-chloro-3-methylphenyl)-3-(2,6-dichlorophenyl)-prop-2-en-1-one
1-(4-chloro-3-methylphenyl)-3-(2-chlorophenyl)prop-2-en-1-one
3-(2-chloro-6-fluorophenyl)-1-(4-chloro-3-methylphenyl)prop-2-en-1-one
3-(4-bromo-2-thienyl)-1-(3,4-dichlorophenyl)prop-2-en-1-one
3-(4-bromo-2-thienyl)-1-(4-chloro-3-methylphenyl)prop-2-en-1-one
3-(4-bromo-2-thienyl)-1-(4-fluorophenyl)prop-2-en-1-one
3-(4-bromo-2-thienyl)-1-(4-chlorophenyl)prop-2-en-1-one
1-(4-chlorophenyl)-3-(2,4-dichlorophenyl)prop-2-en-1-one
3-(1,3-benzodioxol-5-yl)-1-(4-bromophenyl)prop-2-en-1-one
3-(3-phenoxy-2-thienyl)-1-(2-thienyl)prop-2-en-1-one
3-(3-bromo-4-methoxyphenyl)-1-phenylprop-2-en-one
3-(3,4-dichlorophenyl)-1-(2-nitrophenyl)prop-2-en-1-one
1-(4-chlorophenyl)-3-(3,4-dichlorophenyl)prop-2-en-1-one
1-(4-chlorophenyl)-3-(3,5-dichloro-2-hydroxyphenyl)prop-2-en-1-one
1-(2-chlorophenyl)-3-(3,5-dichloro-2-hydroxyphenyl)prop-2-en-1-one
3-(4-chlorophenyl)-1-(2,6-dichlorophenyl)prop-2-en-1-one
1-(4-bromophenyl)-3-(4-chlorophenyl)prop-2-en-1-one
1-(2-chlorophenyl)-3-(2,6-dichlorophenyl)prop-2-en-1-one
1-(4-chlorophenyl)-3-(2,6-dichlorophenyl)prop-2-en-1-one
3-(2,6-dichlorophenyl)-1-(4-methoxyphenyl)prop-2-en-1-one
3-(4-chloro-1-methyl-1H-pyrazol-3-yl)-1-[4-(trifluoromethyl)phenyl]prop-2-en-1-one
3-(2,4-dichlorophenyl)-1-(2-methylphenyl)prop-2-en-1-one
3-(2,6-dichlorophenyl)-1-(2-methylphenyl)prop-2-en-1-one
3-(3,4-dichlorophenyl)-1-(2-methylphenyl)prop-2-en-1-one
3-(5-bromo-2-hydroxyphenyl)-1-(3-methylphenyl)prop-2-en-1-one
3-(5-bromo-2-hydroxyphenyl)-1-(4-methylphenyl)prop-2-en-1-one
3-(2,4-dichlorophenyl)-1-(3-methylphenyl)prop-2-en-1-one
3-(2,4-dichlorophenyl)-1-(4-methoxyphenyl)prop-2-en-1-one
1-[4-amino-2-(methylthio)-1,3-thiazol-5-yl]-3-(4-chlorophenyl)prop-2-en-1-one
1-(4-chlorophenyl)-3-[4-(trifluoromethyl)phenyl]prop-2-en-1-one
1-benzo[b]thiophen-3-yl-3-(4-chlorophenyl)prop-2-en-1-one
1,3-di(5-nitro-3-thienyl)prop-2-en-1-one
1-(4-bromophenyl)-3-(3,5-difluorophenyl)prop-2-en-1-one; and
3-(3,5-difluorophenyl)-1-(3-nitrophenyl)prop-2-en-1-one.

In addition to the above, the following compounds are also activators of HGF/SF pathways useful for the purposes herein:
1-(methylsulfonyl)-5-(2-(2-thienyl)vinyl)-1H-pyrazole
2,2-dimethyl-1-(5-(2-(2-thienyl)vinyl)-1H-pyrazole-1-yl)propan-1-one
N-methyl-5-(2-(2-thienyl)vinyl)-1H-pyrazole-1-carboxamide
(4-chlorophenyl)(5-(3-phenylisoxazol-5-yl)-1H-pyrazol-1-yl)methanone
(4-chlorophenyl)(5-(5-(2-thienyl)-2-thienyl)-1H-pyrazol-1-yl)methanone
(4-(2-chloro-6-fluorobenzyl)-3,5-dimethyl-1H-pyrazol-1-yl)(4-chlorophenyl)methanone
(4-chlorophenyl)(5-(methylthio)-3-(4-phenoxyphenyl)-1H-pyrazol-1-yl)methanone
(4-chlorophenyl)(3,5-dimethyl-4-((1-methyl-1H-imidazol-2-yl)thio)-1H-pyrazol-1-yl)methanone
N1-phenyl-5-(2-(2-thienyl)vinyl)-1H-pyrazole-1-carboxamide
(4-chlorophenyl)(5-(2-(5-(2-thienyl)-2-thienyl)-4-methyl-1,3-thiazol-5-yl)-1H-pyrazol-1-yl)methanone
(5-benzhydryl-1H-pyrazol-1-yl)(4-chlorophenyl)methanone
N1-(4-chlorophenyl)-5-(2-(2-thienyl)vinyl)-1H-pyrazole-1-carboxamide methyl 1-(4-chlorobenzoyl)-5-isoxazol-5-yl-3-methyl-1H-pyrazole-4-carboxylate
2-chloro-6-(4-(1-(4-chlorobenzyl)-1H-pyrazol-5-yl)phenoxy)benzonitrile
4(5-chlorobenzo (b) thiophen-3-yl)-1-(2chlorophenyl)sulfonyl)-3,5dimethyl-1-H-pyrazole
4-(2,6-dichlorobenzyl)-3-methyl-1-phenyl-1H-pyrazol-5-ol
3-methyl-4-(2-methylallyl)-1-(phenylsulfonyl)-1H-pyrazol-5-ol
[3-(2,6-difluorophenyl)-4-ethyl-1H-pyrazol-1-yl](2-thienyl)methanone
4-[(5-chloro-1-benzothiophen-3-yl)methyl]-N,3,5-trimethyl-1H-pyrazole-1-carboxamide
3-(2,6-difluorophenyl)-4-ethyl-1H-pyrazole
N1-(3-chlorophenyl)-4-[(5-chlorobenzo[b]thiophen-3-yl)methyl]-3,5-dimethyl-1H-pyrazole-1-carboxamide
{4-[(5-chlorobenzo[b]thiophen-3-yl)methyl]-3,5-dimethyl-1H-pyrazol-1-yl}(4-nitrophenyl)methanone
N1-phenyl-4-[(5-chlorobenzo[b]thiophen-3-yl)methyl]-3,5-dimethyl-1H-pyrazole-1-carboxamide
4-[(5-chloro-1-benzothiophen-3-yl)methyl]-N-(2,4-dichlorophenyl)-3,5-dimethyl-1H-pyrazole-1-carboxamide
1-[3-(2,6-difluorophenyl)-4-ethyl-1H-pyrazol-1-yl]-2,2-dimethylpropan-1-one
(4-(2-chloro-6-fluorobenzyl)-3,5-dimethyl-1H-pyrazole-1-yl)(3-(2,6-dichlorophenyl)-5-methylisoxazol-4-yl)methanone
4-(2-chloro-6-fluorobenzyl)-1-((3,4-dichlorophenyl)sulfonyl)-3,5-dimethyl-1H-pyrazole
4-(2-chloro-6-fluorobenzyl)-1,3,5-trimethyl-1H-pyrazole
4-(2-chloro-6-fluorobenzyl)-3,5-dimethyl-1H-pyrazole
(4-bromo-3,5-dimethyl-1H-pyrazol-1-yl)(3-(2,6-dichlorophenyl)isoxazole-4-carbohydrazide)
N′4,5-dimethyl-N′4-(5-nitro-2-pyridyl)-3-(2,6-dichlorophenyl)isoxazole-4-carbohydrazide
N′4-(2-(((2,4-dichlorobenzylidene)amino)oxy)acetyl)-3-(2,6-dichlorophenyl)-5-methylisoxazole-4-carbohydrazide
3,5-di(tert-butyl)-4-(2-chloro-6-fluorobenzyl)-1H-pyrazole
N′4-((2-methyl-1,3-thiazol-4-4-yl)carbonyl)-3-(2,6-dichlorophenyl)-5-methylisoxazole-4-4-carbohydrazide
(4-(2-chloro-6-fluorobenzyl)-3,5-dimethyl-1H-pyrazole-1-yl)(4-chlorophenyl)methanone
Pentaphenylbenzene
1,3,5-triphenylbenzene
(3-Biphenyl) Trimethyl silane
16 methyl-16 Dehydropregnenolone
9-biphenyl-4-ylmethylene-9H-tri-benzo(A,C,E)-cycloheptene
1,1,3-triphenylinedene
3-(4-Bromophenyl)-1-phenylprop-2-en-1-one
3,3-dibromo-1-phenyl-1,2,3,4-tetrahydroquinoline-2,4-dione; and
4-(4-chlorophenyl)-6-(dimethylamino)-2-phenyl-5-pyrimidinecarbonitrile.

Some of the foregoing compounds can comprise one or more asymmetric centers, and thus can exist in various isomeric forms, e.g., stereoisomers and/or diastereomers. Thus, the methods of the invention employing compounds herein may be in the form of an individual enantiomer, diastereomer or geometric isomer, or may be in the form of a mixture of stereoisomers. In certain embodiments, the compounds useful for the practice of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The use of the invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds described herein may be prepared by crystallization of compound of formula (I)-(VI) under different conditions and may exist as one or a combination of polymorphs of compound of general formula (I)-(VI) forming part of this invention. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the uses of present invention encompasses compounds, their derivatives, their tautomeric forms, their stereoisomers, their C(5)-positional isomer their polymorphs, their pharmaceutically acceptable salts their pharmaceutically acceptable solvates and pharmaceutically acceptable compositions containing them. Tautomeric forms of compounds of the present invention include, for example the 3- and 5-substituted pyrazole tautomers of any of the aforementioned disubstituted compounds of general Formula II and related formulas. Likewise, C(5)-positional isomers of the 1,3-disubstituted pyrazoles of general Formula I, III and IV and related formulas are encompassed within the scope of the present invention. Thus, the invention encompasses 1,5-disubstituted pyrazoles.

### Pharmaceutical Compositions

As discussed above this invention provides uses of compounds described herein that have biological properties beneficial in the treatment of demyelinating diseases and trauma to the central nervous system. The methods of the invention are carried out by administering a compound of the invention or a pharmaceutical composition that comprises any one or more of the compounds described herein (or a prodrug, pharmaceutically acceptable salt or other pharmaceutically acceptable derivative thereof), and optionally comprise a pharmaceutically acceptable carrier. In certain embodiments, these compositions optionally further comprise one or more additional therapeutic agents. Alternatively, a compound used in the practice of this invention may be administered to a patient in need thereof in combination with the administration of one or more other therapeutic agents. For example, additional therapeutic agents for conjoint administration or inclusion in a pharmaceutical composition with a compound of this invention may be an approved agent to treat the same or related indication, or it may be any one of a number of agents undergoing approval in the Food and Drug Administration that ultimately obtain approval for the treatment of any disorder related to HGF/SF activity. It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

The proteins, fusion proteins, polypeptides, truncates, and other forms of proteins described herein for therapeutic use generally require parenteral administration or administration to a particular site of desired activity with the body. Methods of preparing such proteins and related molecules is well known in the art; means for expressing human proteins in mammalian expression systems is readily performed; guidance may be found among the various citations herein on HGF/SF and means for protein, DNA and expression vector delivery. The nucleic acid and DNA agents of the invention generally but not necessarily require delivery via an expression system such as a viral or other vector in order to introduce the expression construct into cells of the body, though methods of effectively administering DNA directly are known. The skilled artisan will be amply aware of delivery techniques suitable for delivery of such macromolecules; one such source of guidance can be found in published U.S. patent application 20050136036. For example, the expression vector can comprise a virus or engineered vector derived from a viral genome. Such virus vectors include adenovirus vectors, adenovirus assisted virus (AAV) transfection, retroviral vectors, lentivirus vectors, and others including vaccinia virus, sindbis virus, cytomegalovirus and herpes simplex virus. Furthermore, direct delivery of DNA such as by injection, microinjection, electroporation, calcium phosphate precipitation, DEAE-dextran followed by polyethylene glycol, microprojectile bombardment, agitation with silicon carbide fibers, PEG-mediated transformation of protoplasts, or by desiccation/inhibition-mediated DNA uptake are known in the art and may be applied to the teachings hereto. These methods are well known and readily adopted by a skilled artisan in the practice of the present invention.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds useful in the practice of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood, or N-demethylation of a compound of the invention where R¹ is methyl. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions used in the practice of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut (peanut), corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

### Research Uses, Clinical Uses, Pharmaceutical Uses and Methods of Treatment

In certain embodiments, the method involves the administration of a therapeutically effective amount of the compound or a pharmaceutically acceptable derivative thereof to a subject (including, but not limited to a human or animal) in need of it. Subjects for which the benefits of the compounds of the invention are intended for administration include, in addition to humans, livestock, domesticated, zoo and companion animals.

As discussed above this invention provides novel compounds that have biological properties useful for mimicking or agonizing, HGF/SF activity. It will be appreciated that the compounds and compositions, according to the method of the present invention, may be administered using any amount and any route of administration effective for the treatment of conditions or diseases in which HGF/SF or the activities thereof have a therapeutically useful role. Thus, the expression “effective amount” as used herein, refers to a sufficient amount of agent to modulate HGF/SF activity (e.g., mimic HGF/SF activity), and to exhibit a therapeutic effect. The exact amount required will vary from subject to subject, depending on the species, age, and general condition of the subject, the severity of the infection, the particular therapeutic agent, its mode and/or route of administration, and the like. The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, subcutaneously, intradermally, intra-ocularly, topically (as by creams, lotions, powders, ointments, or drops), buccally, as an oral or nasal spray, or the like, depending on the severity of the disease or disorder being treated. In certain embodiments of the invention, the HGF/SF protein or variant thereof may be administered by a route and a dose and frequency of dosing to provide therapeutic levels to achieve the benefits described herein. Preferred routes for administration of protein agents include intravenous, intraarterial, and subcutaneous. In certain embodiments, the small molecule compounds of the invention may be administered at dosage levels of about 0.001 mg/kg to about 50 mg/kg, preferably from about 0.1 mg/kg to about 10 mg/kg for parenteral administration, or preferably from about 1 mg/kg to about 50 mg/kg, more preferably from about 10 mg/kg to about 50 mg/kg for oral administration, of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

The methods of the invention include means for identifying HGF/SF pathway activating activity in agents and compounds for uses described herein. The examples demonstrate numerous means by which compounds or agents can be evaluated in vitro and in vivo for potential activity and utility in the treatment and prophylaxis of the diseases and conditions herein described.

### Treatment Kit

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions useful in the practice of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### Exemplification

The compounds of this invention and their preparation can be understood further by the examples that illustrate some of the processes by which these compounds are prepared or used. It will be appreciated, however, that these examples do not limit the invention. Variations of the invention, now known or further developed, are considered to fall within the scope of the present invention as described herein and as hereinafter claimed.

### General Description of Synthetic Methods

The practitioner has a well-established literature of small molecule chemistry to draw upon, in combination with the information contained herein, for guidance on synthetic strategies, protecting groups, and other materials and methods useful for the synthesis of the compounds of this invention. Reference is made to the above citations for the preparation of the compounds and their pharmaceutical compositions, such as WO2004/058721 and U.S. Pat. No. 6,589,997.

Guidance for preparing DNA and expression vectors for direct administration, and HGF/SF protein is provided herein above and are readily appreciated by the skilled artisan.

### Biological Activity

The examples provided below demonstrate the effectiveness of the compounds described herein at addressing neurodegenerative diseases such as multiple sclerosis and other demyelinating diseases, as well as traumatic injury to the central nervous system such as brain trauma and spinal cord injury. Furthermore, the examples demonstrate assays that can readily be used to assess biological activity in small molecule compounds as well as modified versions of HGF/SF, such as fragments, fusion polypeptides and muteins thereof, as well as expression vectors and DNA encoding such modified forms of HGF/SF. The ordinary skilled artisan can easily identify activity in such compounds by following the teachings herein and in the documents citer herein, all of which are incorporated in their entireties.

### Example 1

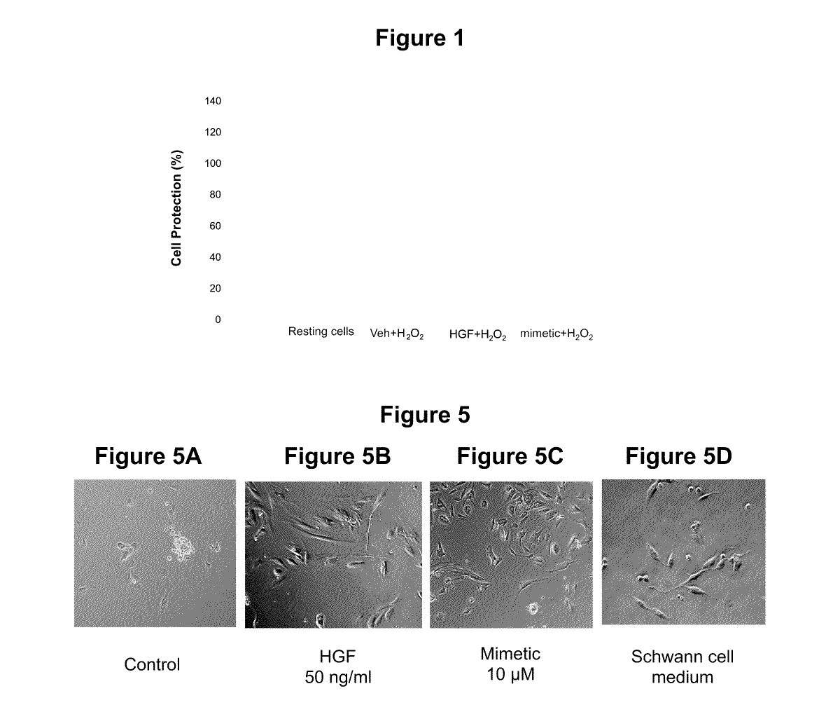

HGF and small molecule HGF/SF mimetic protect Schwann cells against H₂O₂-induced apoptosis. Schwann cells were seeded in 96 well plates at a density of 5000 cells/well, overnight in serum-free media. Cells were treated with 0.5 mM H₂O₂ for 2 hours in the presence of vehicle (Veh), HGF (50 ng/ml) or HGF/SF mimetic (10 micromolar). Cells were assayed for apoptosis using the Apo-ONE Assay kit (Promega). As seen in FIG. 1, treatment with H₂O₂ reduced cell viability to ˜5% of baseline in the vehicle group. By contrast, treatment with HGF or a small molecule mimetic was associated with a substantial improvement in cell viability.

### Example 2

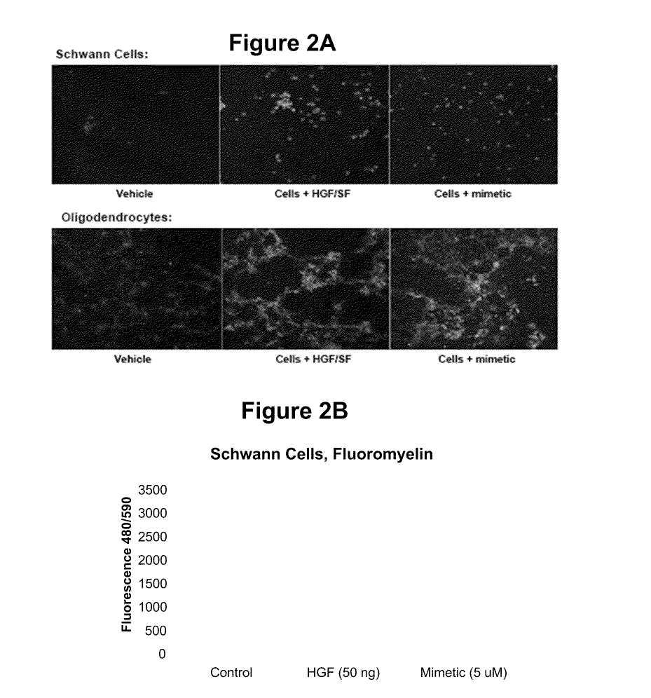

HGF/SF and small molecule HGF/SF mimetic increase myelin production in Schwann cells. It is well-known that the primary function of oligodendrocytes and Schwann cells is myelin production. In a further experiment, HGF/SF and a small molecule mimetic were shown to induce myelin production in oligodendrocytes and Schwann cells, a finding with important therapeutic implications for spinal cord injury (SCI). Cells were seeded in 3 well chamber slides at a density of 50,000 cells/well in serum free media for 24 hours. Then HGF/SF (50 ng/ml) or a small molecule HGF/SF mimetic (5 μM) were added to the same medium and cells incubated for an additional 4 hours. Cells were washed, and fluoromyelin (Molecular Probes) was added to each well (30 min exposure at room temperature), and cells observed under a confocal microscope (Olympus). As seen in FIG. 2A, treatment of cells with HGF or mimetic produced an intense increase in the fluoromyelin signal. To obtain semi-quantitative measurements of this increase in the fluoromyelin signal, a second experiment was performed. Schwann cells were seeded in 6 well plates at a density of 4×10⁵ cells/well and treated with HGF (50 ng/ml) or mimetic (5 uM) for 4 hours. Fluoromyelin staining was performed as described above and relative fluorescence was measured using a fluorescence plate reader. As seen in FIG. 2B, treatment of Schwann cells with HGF or mimetic, induced a 3-fold increase in the fluoromyelin signal.

### Example 3

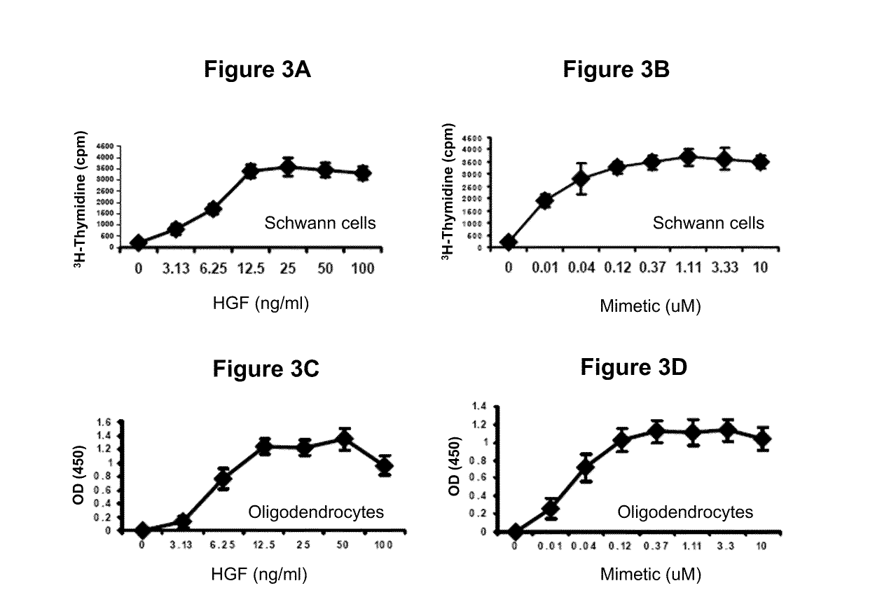

Small molecule HGF/SF agonist mimics HGF/SF's pro-proliferative effects. Oligodendrocytes and Schwann cells are both cells that support neuronal function by ensheathing neuronal axons with myelin. An individual supporting cell comprises a single segment of an axon's myelin sheath and is active in facilitating axonal growth. An important aspect of HGF/SF activity is this growth factor's ability to induce oligodendrocyte and Schwann cell proliferation. In order to evaluate small molecule HGF/SF mimetics for potential HGF/SF-like bioactivity, rat RSC96 Schwann cells (ATCC, Manassas, Va.) or mouse primary oligodendrocytes (Celprogen, San Pedro, Calif.) were seeded in 96-well plates at 5000 cells/well in serum-free medium for 16 hours. Cells were treated with test compound or HGF/SF (positive control) for 16-24 hours. [³H]-thymidine was added to the medium with Schwann cells, WST1 cell proliferation reagent (Roche, Indianapolis, Ind.) was added to oligodendrocytes, and incubation was continued for another 4-5 hours. Schwann cells were washed with PBS and harvested. [³H]-thymidine incorporation was determined and used as a measure of proliferation (increased DNA synthesis). Cells with WST1 reagent were read using a plate reader at an OD of 490 nm. Small molecule HGF/SF mimetic and HGF/SF produced similar effects on cell proliferation, indicating that both HGF/SF and the mimetic activate Schwann cells (FIGS. 3A and 3B) and oligodendrocytes (FIGS. 3C and 3D) in vitro. A similar activation of cell proliferation by the mimetic and HGF/SF was shown for neuronal rat PC-12 cells (ATCC) and also for epithelial and endothelial cells.

### Example 4

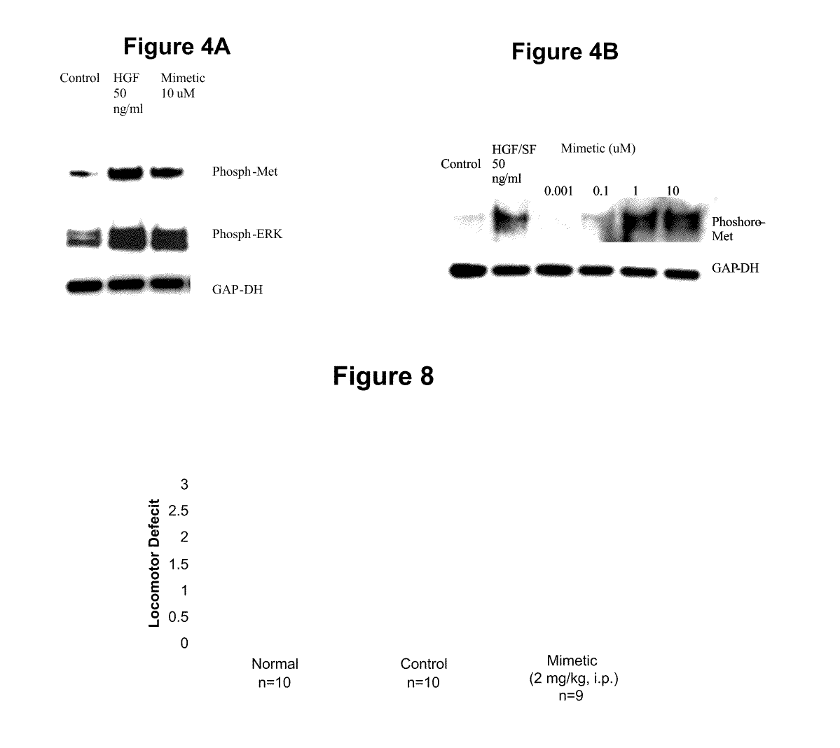

Phosphorylation of the Met Receptor In Vitro. The ability of small molecule HGF/SF mimetics to stimulate Met phosphorylation in Schwann cells, a hallmark of HGF/SF activity, suggested that the primary biologic activities of these compounds were similar to those of HGF/SF. Therefore, the ability of HGF/SF and a mimetic to induce phosphorylation of the HGF/SF receptor, Met, was compared. Schwann cells were incubated with either HGF/SF (50 ng/ml) or mimetic (10 micromolar) for 30 minutes. Western blot data shown in FIG. 4A indicate that the phosphorylation of Met by the mimetic is comparable to HGF/SF, and that the mimetic also has an effect on Erk phosphorylation, which is downstream of in the Met kinase pathway. Similar results were obtained for both human umbilical vein endothelial cells (HUVEC; Cambrex, Walkersville, Md.), suggesting specificity of the mimetic for the HGF/SF/Met intracellular signaling cascade, but not for a specific cell type. The mimetic also phosphorylates the Met receptor in neuronal cells in a dose dependent matter (FIG. 4B). In this experiment, human cortical neuron cells (HCN-2; ATCC) were incubated with either HGF/SF (50 ng/ml) or mimetic (at concentrations as indicated) for 30 minutes prior to Western blot analysis using phosphoro-Met antibodies.

The results mentioned above and shown in FIGS. 3A-D demonstrate that the compound is mitogenic for oligodendrocytes and Schwann cells and can induce cell proliferation in a dose-dependent matter. In contrast, neither the mimetic nor HGF/SF activated mitogenesis in fibroblasts lacking the Met receptor. A similar result was obtained in smooth muscle cells, which also lack the Met receptor. FIG. 4A demonstrates that the mimetic activates the Met pathway in Schwann cells, resulting in downstream activation of Erk kinase. To determine whether mimetic-mediated Met phosphorylation induces similar intracellular signaling cascades as HGF/SF, and to assess whether these signaling events convey biologic activity, experiments were carried out using an Akt inhibitor and the phosphoinositide3-kinase inhibitor Wortmannin. Both the Akt inhibitor and Wortmannin prevented mimetic- and HGF/SF-induced endothelial cell proliferation, suggesting that the mimetic and HGF/SF exert their biologic effects through similar intracellular signaling pathways. These data demonstrate a specificity of the mimetic for the Met pathway.

### Example 5

HGF/SF and HGF/SF mimetic activate neuronal axon growth. In this experiment, the ability of HGF/SF and mimetic to activate axonal growth was assessed. Human neurons (ATCC) were seeded in 6-well plates (1000 cell/well) in media containing 1% FBS. Cells were treated with HGF/SF (50 ng/ml) or mimetic (5 micromolar) for 48 hours. Results (FIG. 5) show that both the mimetic and HGF/SF activate neuronal axon growth. Cell shape, size, and density are clearly different in the presence of HGF/SF and mimetic compared with control. After 48 hours, HGF/SF and mimetic activate neuronal cell proliferation resulting in the increased cell density observed. In one experiment, neurons were incubated with conditioned media from Schwann cells (FIG. 5, right field). In this assay, cell density did not increase compared with control. Thus, secreted growth factors present in Schwann cell media are ineffective (or are at least effects are below the level of detection) as cell mitogens. Nevertheless, these growth factors significantly contribute to axonal growth and guidance.

### Example 6

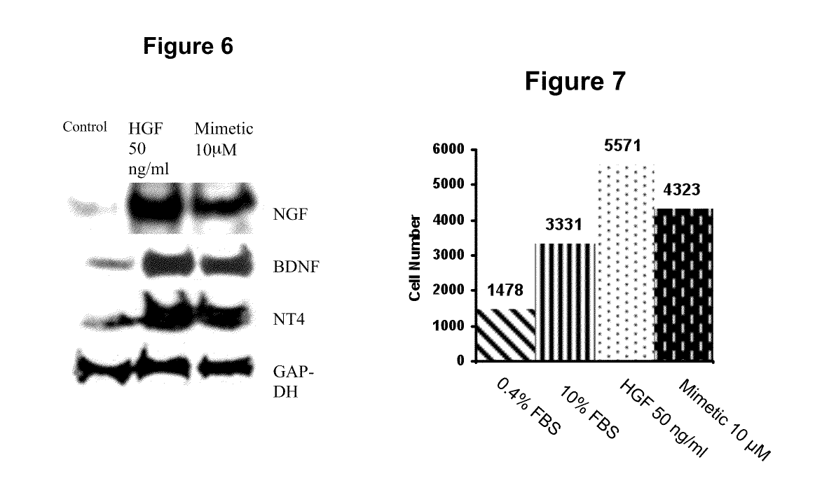

HGF/SF- and HGF/SF mimetic-activated Schwann cells show increased expression of neurotrophic growth factors. In an effort to better understand the beneficial effects of HGF/SF and mimetics on axon growth and neuronal survival, Schwann cells were treated with HGF/SF or mimetic and protein extracts analyzed by Western blot using antibodies against a number of different cytokines The results shown in FIG. 6 demonstrate that Schwann cells activated with HGF/SF or mimetic over-express NGF, BDNF, and NT-4 cytokines Thus, neurotrophic actions of mimetic may be also mediated by stimulation of glial growth factor production.

### Example 7

HGF/SF mimetic recapitulates HGF/SF activity in Schwann cells. To compare the direct effects of HGF/SF mimetic and HGF/SF on cell migration, an in vitro migration assay was employed using a Migration Chamber (BD Biosciences). 50,000 cells were seeded in the inner chamber for 22 hours in the presence of 0.4% or 10% FBS, HGF/SF, or mimetic. Cell number was quantified with 4.5 mg/ml Calcein using a fluorescence plate reader. FIG. 7 demonstrates that both mimetic and HGF/SF similarly increase Schwann cell migration.

### Example 8

Mouse model of multiple sclerosis. Experimental autoimmune encephalomyelitis (EAE) was induced by immunization of female B6 mice with 200 μg MOG 35-55, emulsified in CFA containing 1 mg/ml M. tuberculosis on days 0 and 10 subcutaneously in the hind flank. Three groups of 10 animals were studied: untreated, vehicle-treated controls and HGF/SF mimetic (2 mg/kg/day). The drug administration started immediately after the second injection of peptide. Disease severity was monitored in the blind scoring by two scientists and according to the following scale: 0, no disease; 1, flaccid tail; 2, hind limb weakness; 3, hind limb paralysis; 4, forelimb weakness; 5, moribund. The scores are shown in FIG. 8. The result shows significant recovery effect from developing MS in this animal model. Taken together, these indicate that HGF and HGF mimetics have significant therapeutic potential for treatment of multiple sclerosis.

### Example 9

HGF/SF mimetic improves neurological recovery in spinal cord ischemic injury rabbit model. New Zealand white rabbits were anesthetized with ketamine 20 mg/kg IP, and isoflurane inhalation. Body temperature was maintained at 37±1° C. with a heating blanket. Spinal cord ischemia was produced in all rabbits. Briefly, under clean operative technique, a midline, vertical abdominal laparotomy of 6-7 cm long was made and the intrarenal aorta isolated. An arterial clip was used to cross-clamp the aorta caudal to the left (lower) renal artery. In the rabbit, this corresponds to the second lumbar vertebrae (L 2) level. The aorta was cross-clamped for one hour, then the arterial clip removed to allow the blood reperfusion for 7 days. Immediately after removal of the arterial clip, the abdominal incision was closed in layer with 4.0 silk suture. The wound was sutured closed. Rabbits were returned to their cages with food and water to recover for one week. Treatment was carried out blinded. Each rabbit received 2 mg/kg HGF/SF mimetic (100 ul intravenously via ear vein) or Vehicle once a day for 7 days and there were 10 animals in each treatment group. Initial treatment was given immediately after cross-clamping the abdominal aorta.

Neurological evaluation. After ischemic injury, motor function was tested every day for one week. Two hind limbs were tested in each rabbit for motor deficit according to a modified Tarlov's criteria by an investigator blinded to the experimental procedures. Motor function was graded from 0-5. Grade 0: complete recovery, rabbit is able to hop normally (the two hind limbs are able to leave the ground simultaneously when walking) Grade 1: rabbit is able to hop, but hop wobbly and may fell aside occasionally when hopping. Grade 2: able to stand, but unable to hop; grade 3: good movement of the hind limbs, but unable to stand; Grade 4: spastic paraplegia with slight movement of the hind limbs; grade 5: spastic paraplegia with no movement to the hind limbs.

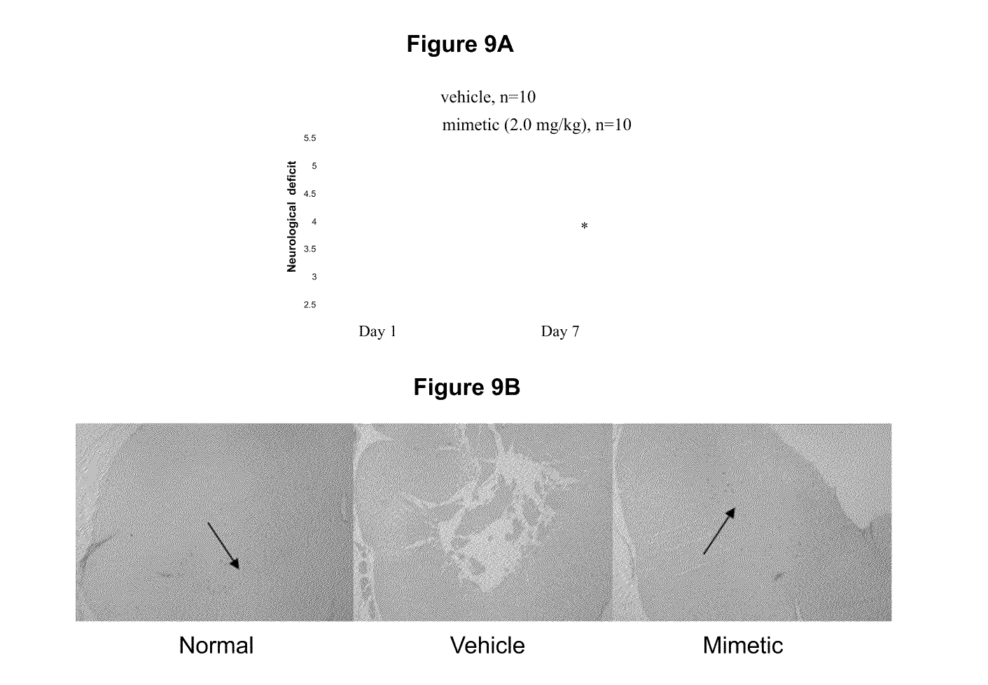

As shown in FIG. 9A, on day 7, the mean neurological deficit score is significantly attenuated in the mimetic-treated animals, p<0.05.

Hematoxylin-eosin (HE) staining. After neurological evaluation, rabbits in each group were euthanized with an overdose of pentobarbital and perfused with 10% buffered formalin through the heart. The spinal cords from low thoracic level to low lumbar level were rapidly harvested by multiple laminectomies and fixed in a 10% buffered formalin solution for one week. Lumbar spinal cord (L2-L6) segments were embedded in paraffin. Sections of 6 μm in thickness were cut and stained with hematoxylin-eosin. FIG. 9B shows that architectural morphology of gray matter is normal, and neurons are well preserved in mimetic-treated group compared with control. Arrows highlight the presence of neurons.

The above data demonstrate effectiveness of HGF/SF and small molecule mimetics for the activation of Schwann cell proliferation, phosphorylation of Met in Schwann cells and human cortical neurons, activation Schwann cell migration, inducing overexpression of neurocytokines and stimulation of neuronal axonal growth. These effects translate in disease models of multiple sclerosis (experimental autoimmune encephalitis) and spinal cord ischemic injury into significant benefits in vivo to the pathologies underlying these conditions, and thus to the treatment of these diseases and conditions in humans and other animals, as non-limiting examples of benefits in general in myelin-related diseases and trauma to the central nervous system.

What is claimed is:

1. A method for treating or preventing a demyelinating disease or a sequela of central nervous system trauma in a mammal comprising administering to said mammal a hepatocyte growth factor/scatter factor pathway activator.

2. The method of claim 1 wherein the demyelinating disease is multiple sclerosis or a hereditary neurodegenerative disorder.

3. The method of claim 1 wherein the central nervous system trauma is traumatic brain injury or spinal cord injury.

4. The method of claim 1 wherein the hepatocyte growth factor/scatter factor pathway activator is a compound or a pharmaceutical composition comprising a compound having the structure: 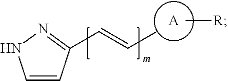
tautomer thereof; or pharmaceutically acceptable derivative thereof;
wherein m is an integer from 1-3;
A represents an optionally substituted aromatic or non-aromatic 5-6 membered monocyclic ring, optionally containing 1-4 heteroatoms selected from N, O or S; or an optionally substituted aromatic or non-aromatic 8-12 membered bicyclic ring, optionally containing 1-6 heteroatoms selected from N, O or S; and
R is one or more substituents selected from the group consisting of hydrogen, halogen, hydroxyl, —NO₂, —CN, an optionally substituted aliphatic, heteroaliphatic, aromatic, heteroaromatic moiety; —OR^{R}, —S(═O)ₙR^{d}, —NR^{b}R^{c}, and —C(═O)R^{a}; wherein n is 0-2, R^{R} is an optionally substituted aliphatic, heteroaliphatic, aromatic, heteroaromatic moiety;
R^{a}, for each occurrence, is independently selected from the group consisting of hydrogen, hydroxy, optionally substituted aliphatic, heteroaliphatic, aryl and heteroaryl;
R^{b} and R^{c}, for each occurrence, are independently selected from the group consisting of hydrogen; hydroxy; SO₂R^{d}; optionally substituted aliphatic, heteroaliphatic, aryl and heteroaryl;
R^{d}, for each occurrence, is independently selected from the group consisting of hydrogen; —N(R^{e})₂; optionally substituted aliphatic, aryl and heteroaryl; and
R^{e}, for each occurrence, is independently hydrogen or optionally substituted aliphatic.

5. The method of claim 4 wherein the compound has the structure: 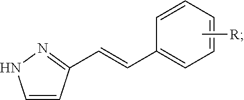
tautomer thereof; or a prodrug, salt, hydrate, or ester thereof;
wherein R is one or more substituents selected from the group consisting of halogen; hydroxy; nitro; CN; aryl; heteroaryl; —C(═O)R^{a}; —NR^{b}R^{c}; —S(O)ₙR^{d} where n=0-2; C₁₋₆alkoxy optionally substituted with one or more substituents independently selected from halogen and C₁₋₆ alkyl; an optionally substituted fused aromatic or non-aromatic 5-6-membered monocyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂;
wherein each occurrence of R^{a} is independently selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
each occurrence of R^{b} and R^{c} is independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
each occurrence of R^{d} is independently selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
each occurrence of R^{e} is independently hydrogen or C₁₋₆ alkyl.

6. The method of claim 4 wherein said compound has the structure: 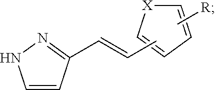
tautomer thereof; or a prodrug, salt, hydrate, or ester thereof;
wherein X is O, S or NR^{N} wherein R^{N} is hydrogen, alkyl, heteroalkyl, aryl, heteroaryl, -(alkyl)aryl, -(alkyl)heteroaryl, acyl or a nitrogen protecting group; and
R is one or more substituents selected from the group consisting of hydrogen, halogen; hydroxy; nitro; CN; aryl; heteroaryl; —C(═O)R^{a}; —NR^{b}R^{c}; —S(O)ₙR^{d} where n=0-2; C₁₋₆alkoxy optionally substituted with one or more substituents independently selected from halogen and C₁₋₆ alkyl; an optionally substituted fused aromatic or non-aromatic 5-6-membered monocyclic ring optionally containing 1-3 heteroatoms selected from the group consisting of N, O, and S; and C₁₋₆ alkyl, C₂₋₆ alkenyl, C₂₋₆ alkynyl, or C₃₋₆ cycloalkyl, each independently optionally substituted with one or more substituents independently selected from halogen, hydroxy, C₁₋₅ alkoxy, nitro, and N(R^{e})₂;
wherein each occurrence of R^{a} is independently selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
each occurrence of R^{b} and R^{c} is independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
each occurrence of R^{d} is independently selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
each occurrence of R^{e} is independently hydrogen or C₁₋₆ alkyl.

7. The method of claim 4 wherein said compound has the structure: 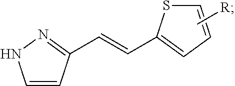
tautomer thereof; or a prodrug, salt, hydrate, or ester thereof.

8. The method of claim 4 wherein said compound has the structure: 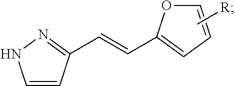
tautomer thereof; or a prodrug, salt, hydrate, or ester thereof.

9. The method of claim 4 wherein said compound has the structure: 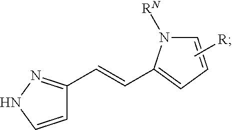
tautomer thereof; or a prodrug, salt, hydrate, or ester thereof;
wherein R^{N} is hydrogen, alkyl, heteroalkyl, aryl, heteroaryl, -(alkyl)aryl, -(alkyl)heteroaryl, acyl or a nitrogen protecting group.

10. The method of claim 4 wherein said compound is from among:

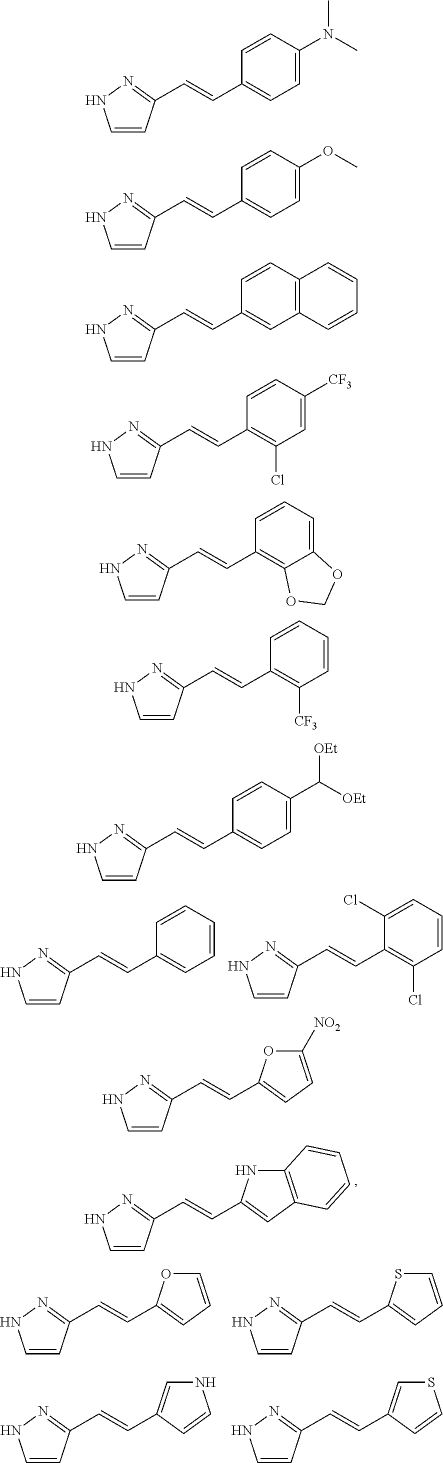

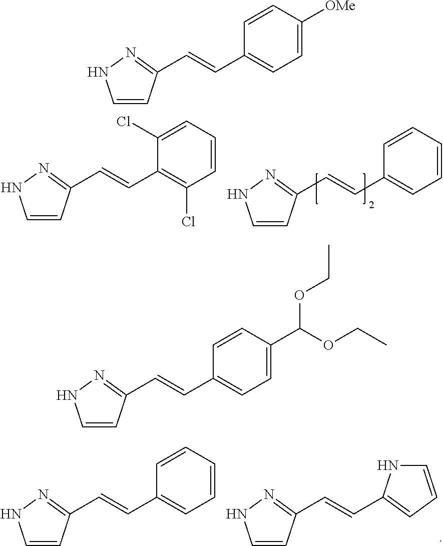

11. The method of claim 1 wherein said hepatocyte growth factor/scatter factor pathway activator is a compound or a pharmaceutical composition comprising a compound having the structure: 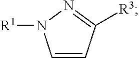
C(5)-positional isomer thereof; or a prodrug, salt, hydrate, or ester thereof;
wherein R¹ is SO₂AL², C(═O)(CH₂)ₘAL², C(═O)OAL², C(═O)NHAL², SO₂Aryl, C(═O)(CH₂)ₘAryl, C(═O)OAryl, C(═O)Oheterocyclic, C(═O)(CH₂)ₘHeterocyclic, or C(═O)NHAryl; wherein m is an integer from 0-3; AL² is an aliphatic or alicyclic moiety; and AL², the aryl and heterocyclic moiety are independently optionally substituted with one or more substituents independently selected from hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃; and
R³ is a cis or trans CHCHAryl, CHCHHeterocyclic, phenoxyphenyl, or a heterocyclic group, wherein the aryl, heterocyclic or phenoxyphenyl moiety may be optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆alkoxy, haloC₁₋₆alkoxy, aryl, heteroaryl and heterocyclyl;
wherein R^{a} is selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl; and
R^{e} is hydrogen or C₁₋₆ alkyl.

12. The method of claim 11 wherein said compound has the structure: 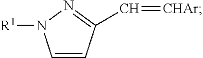
C(5)-positional isomer thereof; or a prodrug, salt, hydrate, or ester thereof;
wherein R¹ is SO₂AL², C(═O)(CH₂)ₘAL², C(═O)OAL², C(═O)NHAL², SO₂Aryl, C(═O)(CH₂)ₘAryl, C(═O)OAryl, C(═O)Oheterocyclic, C(═O)(CH₂)ₘHeterocyclic, or C(═O)NHAryl; wherein m is an integer from 1-3; AL² is an aliphatic or alicyclic moiety; and AL², the aryl and heterocyclic moiety are independently optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl; or COCH₂OC₂H₅OCH₃; and
CHCHAr is a cis or trans CH═CHAryl optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆alkoxy, aryl, heteroaryl and heterocyclyl;
wherein R^{a}, R^{b}, R^{c}, R^{d} and R^{e} are as defined in claim 13.

13. The method of claim 11 wherein said compound has the structure: 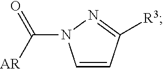
C(5)-positional isomer thereof; or a prodrug, salt, hydrate, or ester thereof;
wherein AR is an optionally fused 3-12 membered aromatic or alicyclic mono- or bicyclic-ring containing 0-3 heteroatoms selected from the group consisting of N, O, and S optionally substituted with one or more substituents independently selected from the group consisting of hydrogen; halogen; hydroxy; nitro; CN; aryl; heteroaryl; heterocycle; carboxy ester; —C(═O)R^{a}, —NR^{b}R^{c}, or —S(O)ₙR^{d} where n=0-2; C₁₋₆alkoxy substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; —NR^{f}R^{g}; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆alkoxy, haloC₁₋₆alkoxy, aryl, heteroaryl and heterocyclyl; and
R³ is a cis or trans CHCHheterocyclic, phenoxyphenyl, or a heterocyclic group, optionally substituted with one or more substituents independently selected from the group consisting of hydrogen; halogen; hydroxy; nitro; CN; aryl; heteroaryl; —C(═O)R^{a}, —NR^{b}R^{c}, or —S(O)ₙR^{d} where n=0-2; C₁₋₆alkoxy optionally substituted with one or more substituents independently selected from halogen and C₁₋₆ alkyl; an optionally substituted fused bicyclic 8-12-membered aromatic or alicyclic ring containing 0-3 heteroatoms selected from the group consisting of N, O, and S; C₁₋₆ alkyl, C₂₋₆ alkenyl, C₂₋₆ alkynyl, or C₃₋₆ cycloalkyl, optionally substituted with one or more substituents independently selected from halogen, hydroxy, C₁₋₅ alkoxy, nitro, and N(R^{e})₂; and further optionally substituted with 1-3 substituents independently selected from the group consisting of —C(═O)R^{a}, —NR^{b}R^{c}, —S(O)ₙR^{d} where n=0-2, hydroxy, C₁₋₆ alkoxy, haloC₁₋₆ alkoxy, aryl, heteroaryl and heterocyclyl;
wherein R^{a} is selected from the group consisting of hydrogen, hydroxy, C₁₋₆ alkyl, C₁₋₆ alkoxy, aryl, heteroaryl, and NR^{b}R^{c}, wherein C₁₋₆ alkyl and C₁₋₆ alkoxy are optionally substituted with one or more substituents independently selected from halogen, hydroxy, C₁₋₅ alkoxy, nitro, and N(R^{e})₂;
R^{b} and R^{c} are independently selected from the group consisting of hydrogen; hydroxy; SO₂R^{d}; C₁₋₆ alkyl optionally substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂;
R^{d} is selected from the group consisting of hydrogen; N(R^{e})₂; C₁₋₆ alkyl optionally substituted with one or more substituents independently selected from halogen, hydroxy, C₁₋₅ alkoxy, nitro, and N(R^{e})₂; aryl and heteroaryl;
R^{e} is hydrogen or C₁₋₆ alkyl; and
R^{f} and R^{g} are independently selected from the group consisting of hydrogen; hydroxy; SO₂R^{d}; C₁₋₆ alkyl substituted with one or more substituents independently selected from halogen, hydroxy, C₁₋₅ alkoxy, nitro, and N(R^{e})₂; C₁₋₆ alkoxy optionally substituted with one or more substituents independently selected from halogen, hydroxy, C₁₋₅ alkoxy, nitro and N(R^{e})₂; aryl optionally substituted with one or more substituents independently selected from halogen, hydroxy, C₁₋₄ alkyl, C₁₋₅ alkoxy, nitro, and N(R^{e})₂; and heteroaryl optionally substituted with one or more substituents independently selected from halogen, hydroxy, C₁₋₄ alkyl, C₁₋₅ alkoxy, nitro, and N(R^{e})₂.

14. The method of claim 11 wherein said compound is from among: 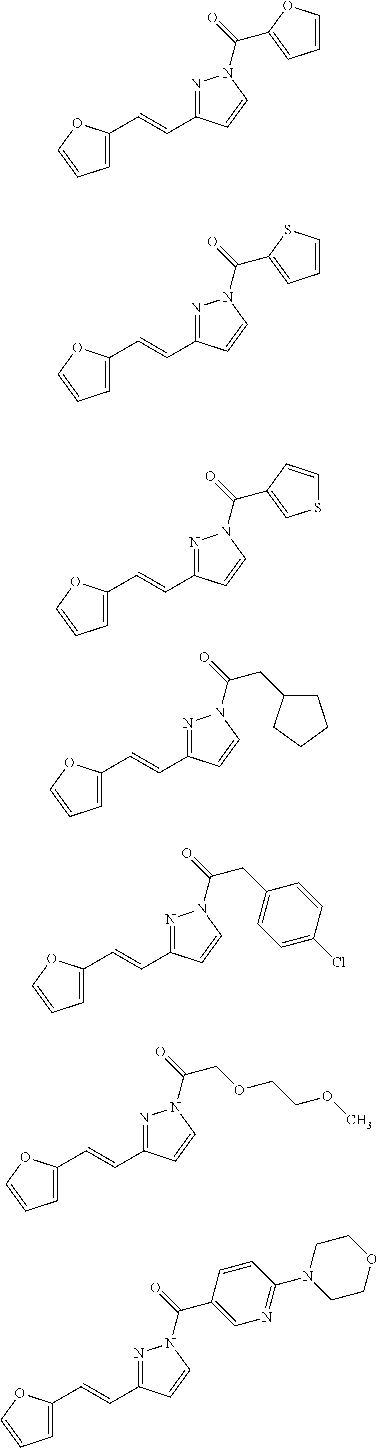 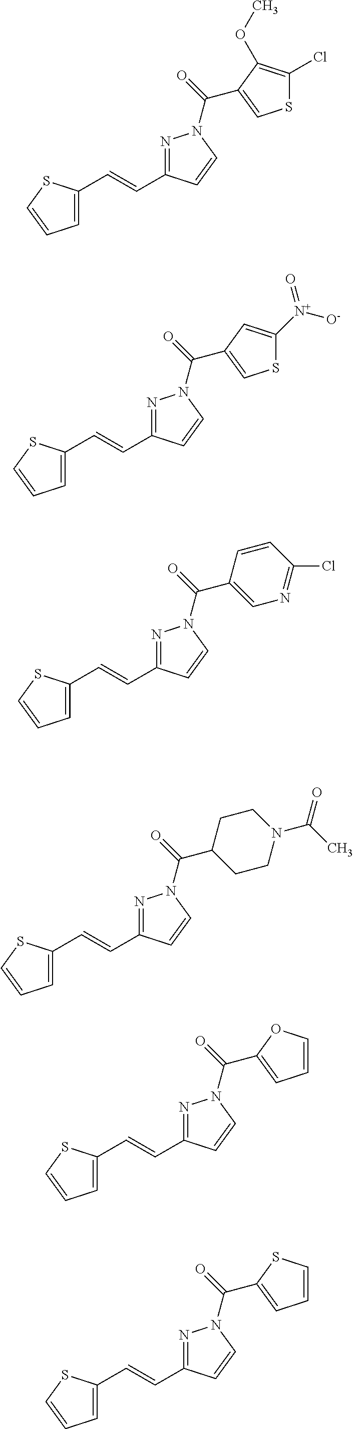 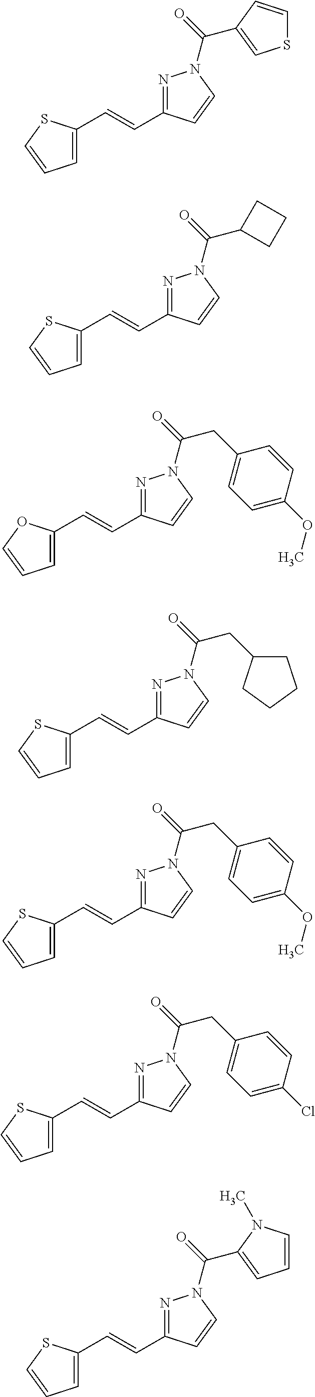 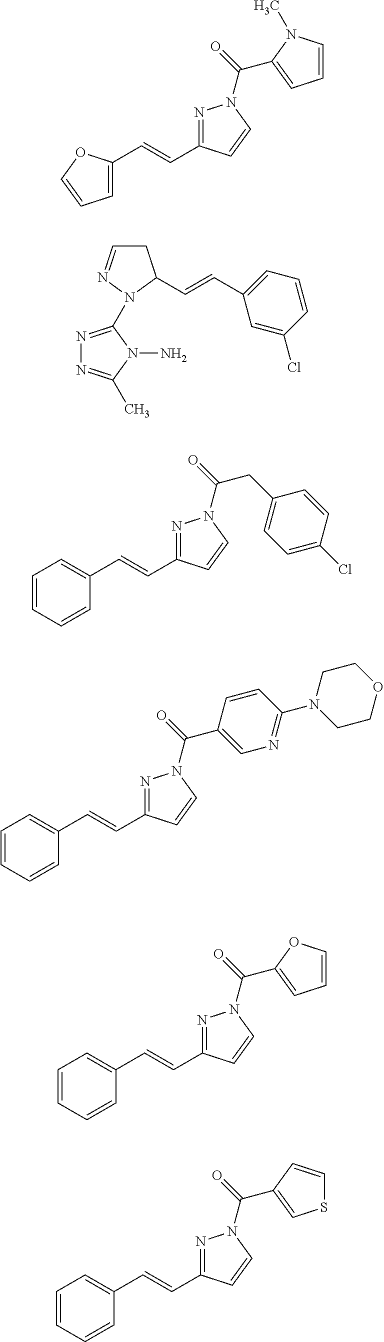 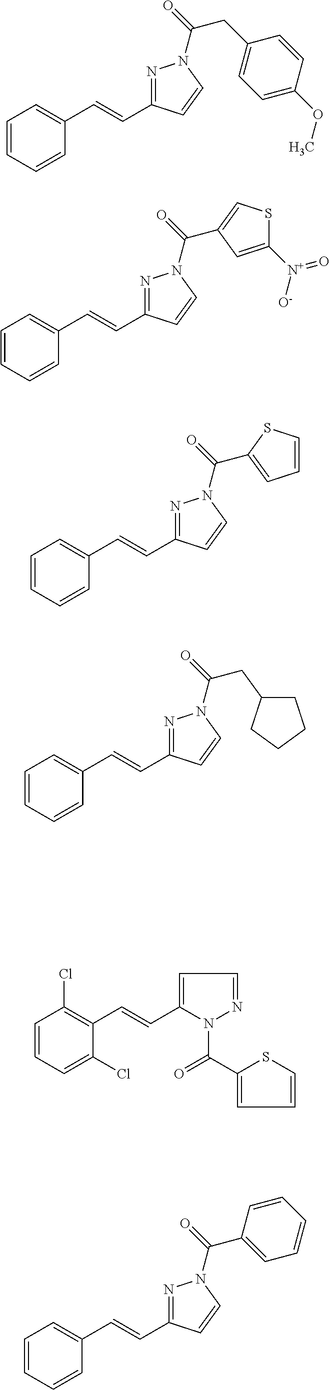 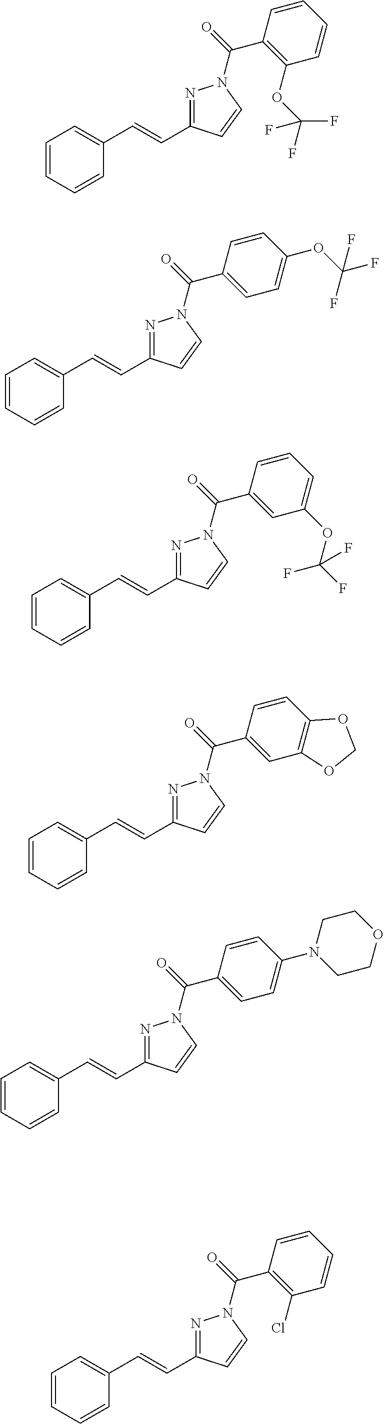 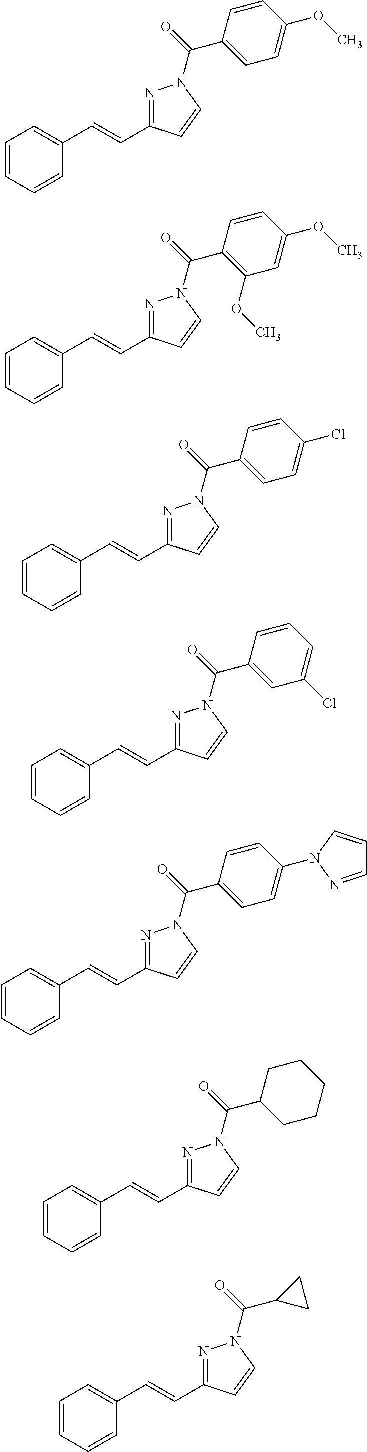 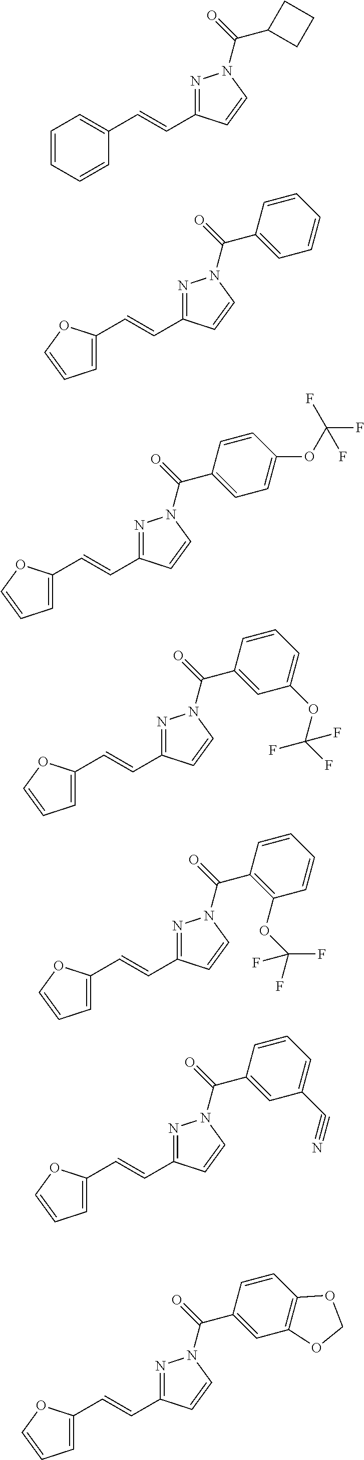 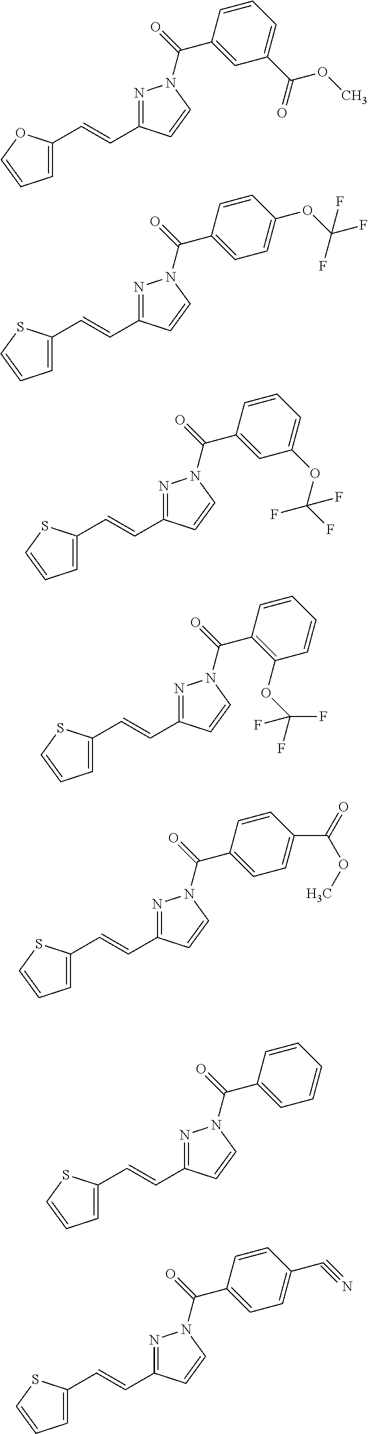 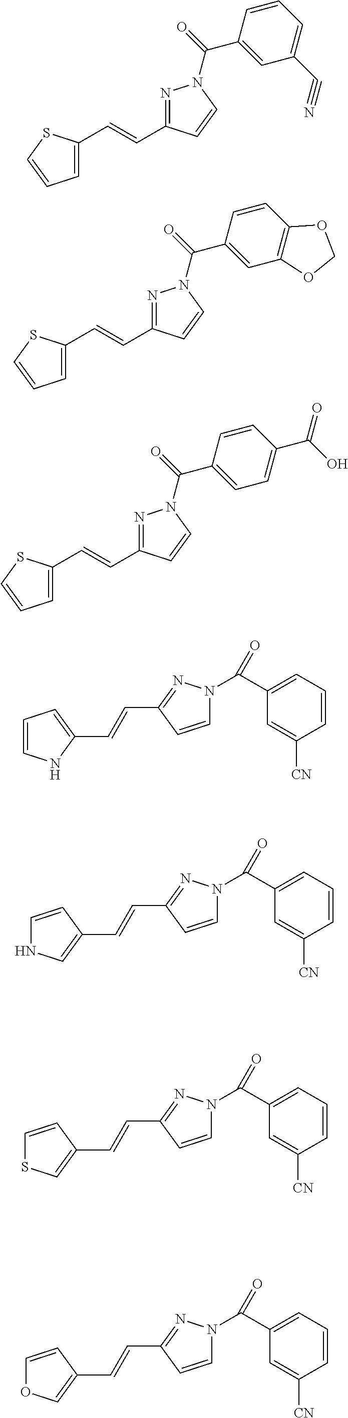 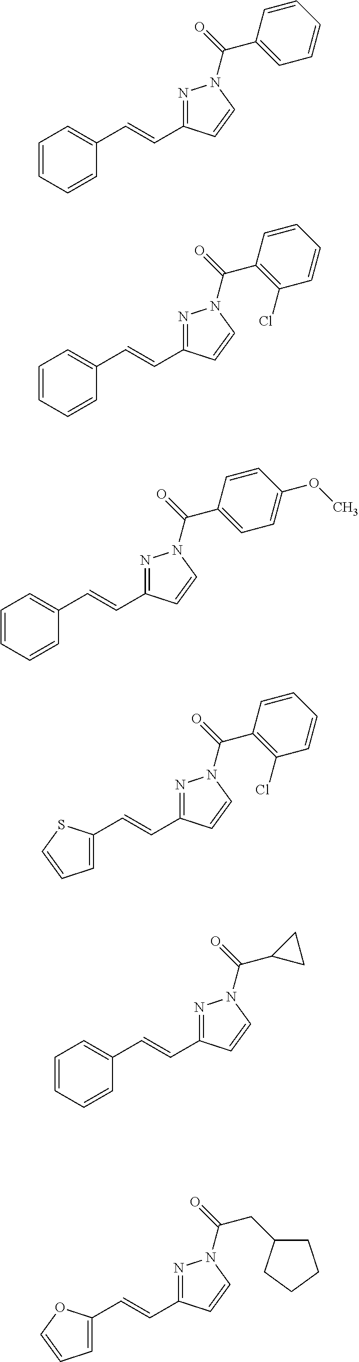 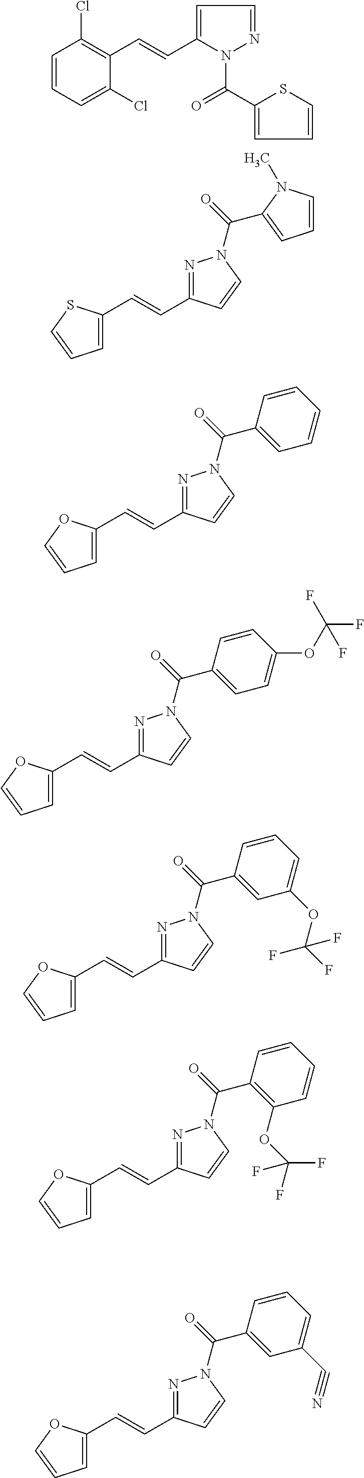 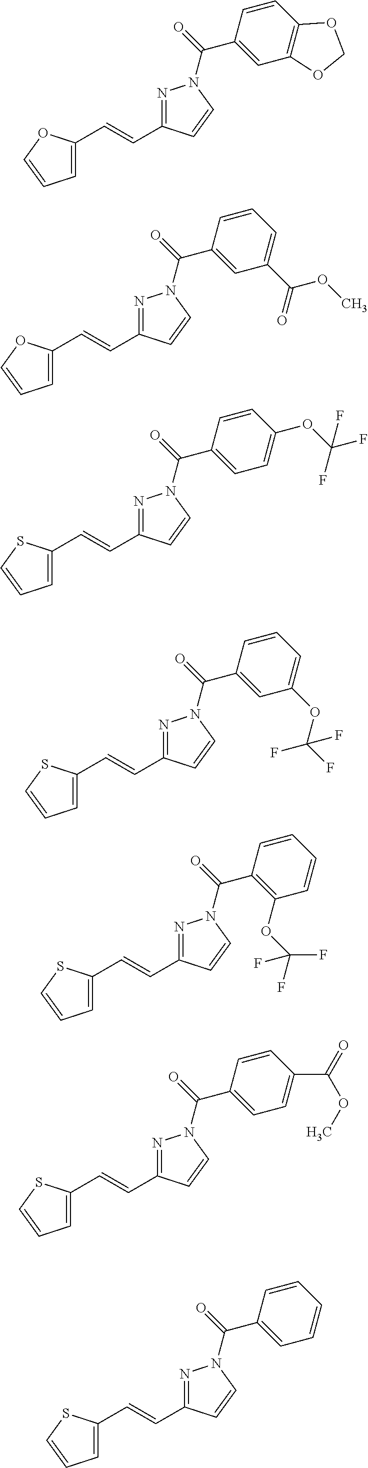 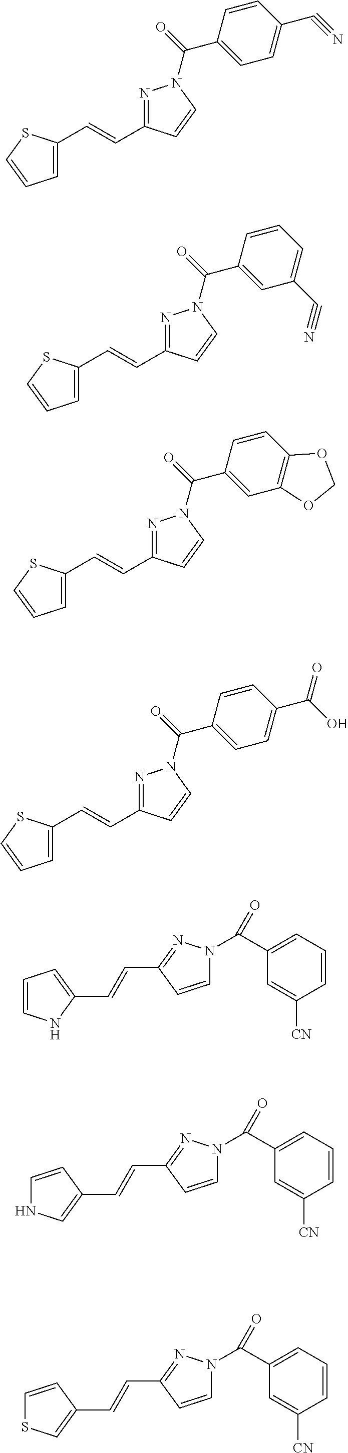 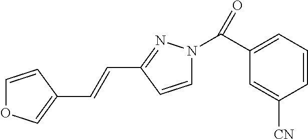
(4-chlorophenyl)[5-(2-(2-thienyl)vinyl)-1H-pyrazol-1-yl]methanone;
1-(methylsulfonyl)-5-(2-(2-thienyl)vinyl)-1H-pyrazole;
2,2-dimethyl-1-(5-(2-(2-thienyl)vinyl)-1H-pyrazole-1-yl)propan-1-one
N-methyl-5-(2-(2-thienyl)vinyl)-1H-pyrazole-1-carboxamide
(4-chlorophenyl)(5-(3-phenylisoxazol-5-yl)-1H-pyrazol-1-yl)methanone
(4-chlorophenyl)(5-(3-(4-chlorophenyl)-5-methylisoxazol-4-yl)-1H-pyrazol-1-yl)methanone
(4-chlorophenyl)(5-(5-(2-thienyl)-2-thienyl)-1H-pyrazol-1-yl)methanone
(2,4-dichlorophenyl)(5-(5-(2,4-difluorophenyl)-2-furyl)-1H-pyrazol-1-yl)methanone
N1-phenyl-5-(2-(2-thienyl)vinyl)-1H-pyrazole-1-carboxamide
(4-chlorophenyl)(5-(2-(5-(2-thienyl)-2-thienyl)-4-methyl-1,3-thiazol-5-yl)-1H-pyrazol-1-yl)methanone
(5-benzhydryl-1H-pyrazol-1-yl)(4-chlorophenyl)methanone
N1-(4-chlorophenyl)-5-(2-(2-thienyl)vinyl)-1H-pyrazole-1-carboxamide
(4-chlorophenyl)(5-(2-methylimidazo(1,2-a)pyridin-3-yl)-1H-pyrazol-1-yl)methanone 2-chloro-6-(4-(1-(4-chlorobenzyl)-1H-pyrazol-5-yl)phenoxy)benzonitrile
1-((4-chlorophenyl)sulfonyl)-5-(2-(2-thienyl)vinyl)-1H-pyrazole

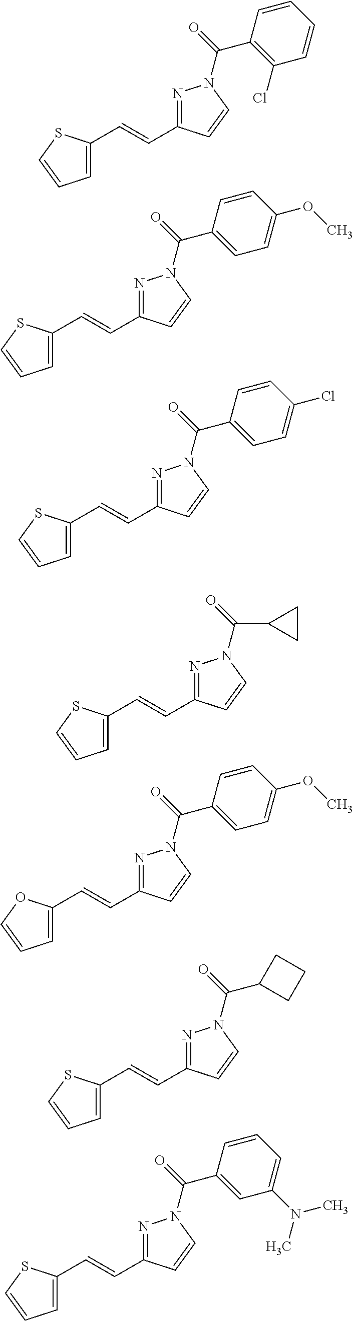

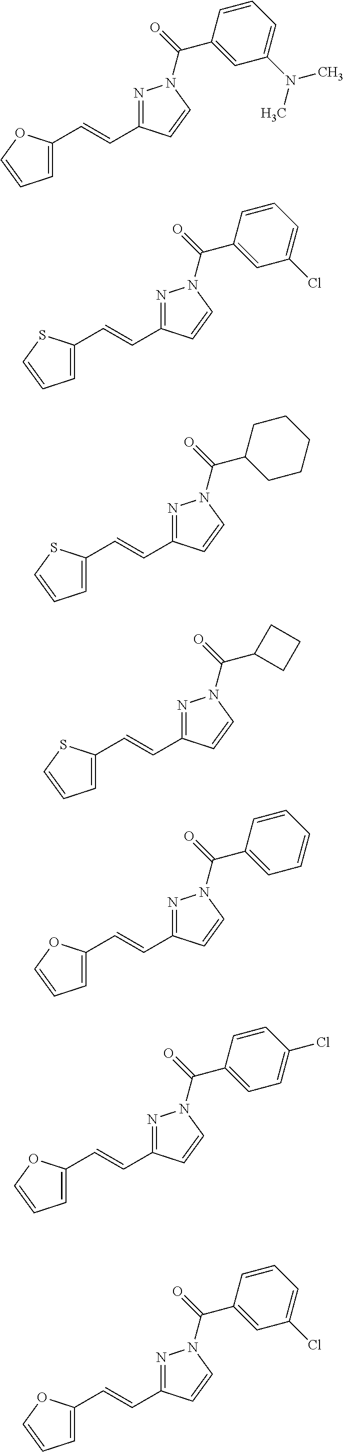

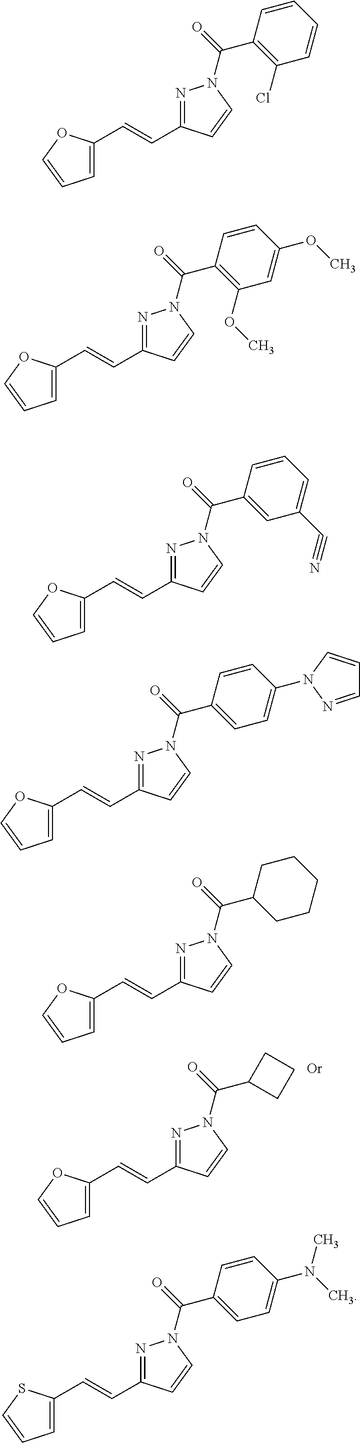

15. The method of claim 1 wherein said hepatocyte growth factor/scatter factor pathway activator is a compound or a pharmaceutical composition comprising a compound having the structure: 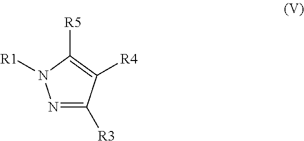
wherein R3 and R5 are independently or together a straight-chain or branched C1-C6 alkyl optionally substituted with a cyano or halogen, halogen, trifluoromethyl or difluoromethyl groups; R1 is hydrogen, methyl, CO-Aryl, SO₂-Aryl, CO-heteroaryl, or CO-alkyl; and R4 is CH₂-Aryl, halogen, arylcarbonylvinyl or S-heteroaryl.

16. The method of claim 15 wherein said compound is from among:
3-(5-chloro-1,3-dimethyl-1H-pyrazol-4-yl)-1-(4-chlorophenyl)prop-2-en-1-one
[4-(2,6-dichlorobenzyl)-3,5-dimethyl-1H-pyrazol-1-yl][3-(2,6-dichlorophenyl)-5-methylisoxazol-4-yl]methanone
(4-(2-chloro-6-fluorobenzyl)-3,5-dimethyl-1H-pyrazole-1-yl)(3-(2,6-dichlorophenyl)-5-methylisoxazol-4-yl)methanone
4-(2-chloro-6-fluorobenzyl)-1-((3,4-dichlorophenyl)sulfonyl)-3,5-dimethyl-1H-pyrazole
4-(2-chloro-6-fluorobenzyl)-1,3,5-trimethyl-1H-pyrazole 4-(2-chloro-6-fluorobenzyl)-3,5-dimethyl-1H-pyrazole
(4-bromo-3,5-dimethyl-1H-pyrazol-1-yl)(3-(2,6-dichlorophenyl)isoxazole-4-carbohydrazide)
3-(4-(2,6-dichlorobenzyl)-3,5-dimethyl-1H-pyrazol-1-yl)propanenitrile
3,5-di(tert-butyl)-4-(2-chloro-6-fluorobenzyl)-1H-pyrazole
(4-(2-chloro-6-fluorobenzyl)-3,5-dimethyl-1H-pyrazole-1-yl)(2,6-dichlorophenyl)methanone
1-(4-(2-chloro-6-fluorobenzyl)-3,5-dimethyl-1H-pyrazole-1-yl)2,2-dimethylpropan-1-one
(4-(2-chloro-6-fluorobenzyl)-3,5-dimethyl-1H-pyrazole-1-yl)(4-chlorophenyl)methanone
(4-(2-chloro-6-fluorobenzyl)-3,5-dimethyl-1H-pyrazole-1-yl)(2-thienyl)methanone; or
(4-chlorophenyl)(3,5-dimethyl-4-((1-methyl-1H-imidazol-2-yl)thio)-1H-pyrazol-1-yl)methanone.

17. The method of claim 1 wherein said hepatocyte growth factor/scatter factor pathway activator is a compound having the general formula VI: 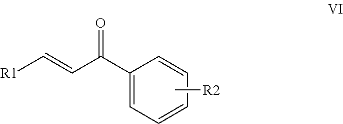
wherein R1 is Aryl or Heteroaryl; and R2 is one or more halogen, nitro, C1 to C4 straight-chained alkyl, branched alkyl, or cycloalkyl, or C1 to C4 alkyloxy groups.

18. The method of claim 17 wherein said compound is from among:
1-(4-chloro-3-methylphenyl)-3-(2,6-dichlorophenyl)-prop-2-en-1-one
1-(4-chloro-3-methylphenyl)-3-(2-chlorophenyl)prop-2-en-1-one
3-(2-chloro-6-fluorophenyl)-1-(4-chloro-3-methylphenyl)prop-2-en-1-one
3-(4-bromo-2-thienyl)-1-(3,4-dichlorophenyl)prop-2-en-1-one
3-(4-bromo-2-thienyl)-1-(4-chloro-3-methylphenyl)prop-2-en-1-one
3-(4-bromo-2-thienyl)-1-(4-fluorophenyl)prop-2-en-1-one
3-(4-bromo-2-thienyl)-1-(4-chlorophenyl)prop-2-en-1-one
1-(4-chlorophenyl)-3-(2,4-dichlorophenyl)prop-2-en-1-one
3-(1,3-benzodioxol-5-yl)-1-(4-bromophenyl)prop-2-en-1-one
3-(3-phenoxy-2-thienyl)-1-(2-thienyl)prop-2-en-1-one
3-(3-bromo-4-methoxyphenyl)-1-phenylprop-2-en-one
3-(3,4-dichlorophenyl)-1-(2-nitrophenyl)prop-2-en-1-one
1-(4-chlorophenyl)-3-(3,4-dichlorophenyl)prop-2-en-1-one
1-(4-chlorophenyl)-3-(3,5-dichloro-2-hydroxyphenyl)prop-2-en-1-one
1-(2-chlorophenyl)-3-(3,5-dichloro-2-hydroxyphenyl)prop-2-en-1-one
3-(4-chlorophenyl)-1-(2,6-dichlorophenyl)prop-2-en-1-one
1-(4-bromophenyl)-3-(4-chlorophenyl)prop-2-en-1-one
1-(2-chlorophenyl)-3-(2,6-dichlorophenyl)prop-2-en-1-one
1-(4-chlorophenyl)-3-(2,6-dichlorophenyl)prop-2-en-1-one
3-(2,6-dichlorophenyl)-1-(4-methoxyphenyl)prop-2-en-1-one
3-(4-chloro-1-methyl-1H-pyrazol-3-yl)-1-[4-(trifluoromethyl)phenyl]prop-2-en-1-one
3-(2,4-dichlorophenyl)-1-(2-methylphenyl)prop-2-en-1-one
3-(2,6-dichlorophenyl)-1-(2-methylphenyl)prop-2-en-1-one
3-(3,4-dichlorophenyl)-1-(2-methylphenyl)prop-2-en-1-one
3-(5-bromo-2-hydroxyphenyl)-1-(3-methylphenyl)prop-2-en-1-one
3-(5-bromo-2-hydroxyphenyl)-1-(4-methylphenyl)prop-2-en-1-one
3-(2,4-dichlorophenyl)-1-(3-methylphenyl)prop-2-en-1-one
3-(2,4-dichlorophenyl)-1-(4-methoxyphenyl)prop-2-en-1-one
1-[4-amino-2-(methylthio)-1,3-thiazol-5-yl]-3-(4-chlorophenyl)prop-2-en-1-one
1-(4-chlorophenyl)-3-[4-(trifluoromethyl)phenyl]prop-2-en-1-one
1-benzo[b]thiophen-3-yl-3-(4-chlorophenyl)prop-2-en-1-one
1,3-di(5-nitro-3-thienyl)prop-2-en-1-one
1-(4-bromophenyl)-3-(3,5-difluorophenyl)prop-2-en-1-one; or
3-(3,5-difluorophenyl)-1-(3-nitrophenyl)prop-2-en-1-one.

19. The method of claim 1 wherein said hepatocyte growth factor/scatter factor pathway activator is from among:
1-(methylsulfonyl)-5-(2-(2-thienyl)vinyl)-1H-pyrazole
2,2-dimethyl-1-(5-(2-(2-thienyl)vinyl)-1H-pyrazole-1-yl)propan-1-one
N-methyl-5-(2-(2-thienyl)vinyl)-1H-pyrazole-1-carboxamide
(4-chlorophenyl)(5-(3-phenylisoxazol-5-yl)-1H-pyrazol-1-yl)methanone
(4-chlorophenyl)(5-(5-(2-thienyl)-2-thienyl)-1H-pyrazol-1-yl)methanone
(4-(2-chloro-6-fluorobenzyl)-3,5-dimethyl-1H-pyrazol-1-yl)(4-chlorophenyl)methanone
(4-chlorophenyl)(5-(methylthio)-3-(4-phenoxyphenyl)-1H-pyrazol-1-yl)methanone
(4-chlorophenyl)(3,5-dimethyl-4-((1-methyl-1H-imidazol-2-yl)thio)-1H-pyrazol-1-yl)methanone
N1-phenyl-5-(2-(2-thienyl)vinyl)-1H-pyrazole-1-carboxamide
(4-chlorophenyl)(5-(2-(5-(2-thienyl)-2-thienyl)-4-methyl-1,3-thiazol-5-yl)-1H-pyrazol-1-yl)methanone
(5-benzhydryl-1H-pyrazol-1-yl)(4-chlorophenyl)methanone
N1-(4-chlorophenyl)-5-(2-(2-thienyl)vinyl)-1H-pyrazole-1-carboxamide
methyl 1-(4-chlorobenzoyl)-5-isoxazol-5-yl-3-methyl-1H-pyrazole-4-carboxylate
2-chloro-6-(4-(1-(4-chlorobenzyl)-1H-pyrazol-5-yl)phenoxy)benzonitrile
4(5-chlorobenzo[b]thiophen-3-yl)-1-(2-chlorophenyl)sulfonyl)-3,5-dimethyl-1-H-pyrazole
4-(2,6-dichlorobenzyl)-3-methyl-1-phenyl-1H-pyrazol-5-ol
3-methyl-4-(2-methylallyl)-1-(phenylsulfonyl)-1H-pyrazol-5-ol
[3-(2,6-difluorophenyl)-4-ethyl-1H-pyrazol-1-yl](2-thienyl)methanone
4-[(5-chloro-1-benzothiophen-3-yl)methyl]-N, 3,5-trimethyl-1H-pyrazole-1-carboxamide
3-(2,6-difluorophenyl)-4-ethyl-1H-pyrazole
N1-(3-chlorophenyl)-4-[(5-chlorobenzo[b]thiophen-3-yl)methyl]-3,5-dimethyl-1H-pyrazole-1-carboxamide
{4-[(5-chlorobenzo[b]thiophen-3-yl)methyl]-3,5-dimethyl-1H-pyrazol-1-yl}(4-nitrophenyl)methanone
N1-phenyl-4-[(5-chlorobenzo[b]thiophen-3-yl)methyl]-3,5-dimethyl-1H-pyrazole-1-carboxamide
4-[(5-chloro-1-benzothiophen-3-yl)methyl]-N-(2,4-dichlorophenyl)-3,5-dimethyl-1H-pyrazole-1-carboxamide
1-[3-(2,6-difluorophenyl)-4-ethyl-1H-pyrazol-1-yl]-2,2-dimethylpropan-1-one
(4-(2-chloro-6-fluorobenzyl)-3,5-dimethyl-1H-pyrazole-1-yl)(3-(2,6-dichlorophenyl)-5-methylisoxazol-4-yl)methanone
4-(2-chloro-6-fluorobenzyl)-1-((3,4-dichlorophenyl)sulfonyl)-3,5-dimethyl-1H-pyrazole
4-(2-chloro-6-fluorobenzyl)-1,3,5-trimethyl-1H-pyrazole
4-(2-chloro-6-fluorobenzyl)-3,5-dimethyl-1H-pyrazole
(4-bromo-3,5-dimethyl-1H-pyrazol-1-yl)(3-(2,6-dichlorophenyl)isoxazole-4-carbohydrazide)
N′4,5-dimethyl-N′4-(5-nitro-2-pyridyl)-3-(2,6-dichlorophenyl)isoxazole-4-carbohydrazide
N′4-(2(((2,4-dichlorobenzylidene)amino)oxy)acetyl)-3-(2,6-dichlorophenyl)-5-methylisoxazole-4-carbohydrazide
3,5-di(tert-butyl)-4-(2-chloro-6-fluorobenzyl)-1H-pyrazole
N′4-((2-methyl-1,3-thiazol-4-4-yl)carbonyl)-3-(2,6-dichlorophenyl)-5-methylisoxazole-4-4 carbohydrazide
(4-(2-chloro-6-fluorobenzyl)-3,5-dimethyl-1H-pyrazole-1-yl)(4-chlorophenyl)methanone
Pentaphenylbenzene
1,3,5-triphenylbenzene
(3-Biphenyl) Trimethyl silane
16 methyl-16 Dehydropregnenolone
9-biphenyl-4-ylmethylene-9H-tri-benzo(A,C,E)-cycloheptene
1,1,3-triphenylinedene
3-(4-Bromophenyl)-1-phenylprop-2-en-1-one
3,3-dibromo-1-phenyl-1,2,3,4-tetrahydroquinoline-2,4-dione; and
4-(4-chlorophenyl)-6-(dimethylamino)-2-phenyl-5-pyrimidinecarbonitrile.

20. The method of claim 1 wherein said hepatocyte growth factor/scatter factor pathway activator is hepatocyte growth factor/scatter factor, or a fragment, fusion polypeptide or mutein thereof.

21. The method of claim 1 wherein said hepatocyte growth factor/scatter factor pathway activator is a nucleic acid or an expression vector comprising a nucleic acid encoding hepatocyte growth factor/scatter factor, a fragment, fusion polypeptide or mutein thereof.
